(19) United States
(12) Patent Application Publication
(10) Pub. No.: US 2019/0051420 A1
(43) Pub. Date: Feb. 14, 2019

(54) EVOLVING CONTEXTUAL CLINICAL DATA ENGINE FOR MEDICAL DATA PROCESSING

(71) Applicant: TeraRecon, Inc., Foster City, CA (US)

(72) Inventors: Tiecheng Zhao, Concord, CA (US); Jeffrey Sorenson, Milwaukee, CA (US); Akio Iwase, Brisbane, CA (US)

(21) Appl. No.: 16/159,655

(22) Filed: Oct. 13, 2018

Related U.S. Application Data
(63) Continuation of application No. 14/711,571, filed on May 13, 2015.
(60) Provisional application No. 61/993,005, filed on May 14, 2014.

(51) Int. Cl. G16H 50/70; G06F 19/00; G16H 10/60; G16H 50/20; G16H 40/63

(52) U.S. Cl. CPC G16H 50/70; G06F 19/00; G16H 40/63; G16H 50/20; G16H 10/60

(57) ABSTRACT

A medical information server receives a signal from a client device representing a first user interaction with first medical data associated with a first medical condition of a patient received from a first medical data server. A data retrieval module accesses a second medical data server to retrieve second medical data of the patient that is related to the first medical data. A data analysis module automatically performs a first analysis on image data of the first medical data to generate image quantitative result and a second analysis on the image quantitative result in view of other medical data of the patient to determine a likelihood of a second medical condition of the patient based on the analysis. A data integrator integrates the second medical data with an analysis result of the analysis to generate and transmit a view of medical information to the client device to be displayed therein.

### RELATED APPLICATIONS

This application claims the benefit of U.S. Provisional Application No. 61/993,005 filed May 14, 2014, which is incorporated by reference herein in its entirety.

### FIELD OF THE INVENTION

Embodiments of the present invention relate generally to medical information processing systems. More particularly, embodiments of the invention relate to medical data processing using an evolving contextual clinical data (ECCD) engine.

### BACKGROUND

The electronic health record (EHR) and/or electronic medical record (EMR) are quickly becoming the standard for capturing, storing and displaying medical information. However, there is also a need to analyze and use medical information from several very different sources. For example, medical information can exist as text or structured data in an EMR, or unstructured data such as a voice recording in a dictation system. Data may exist as a graph or chart such as an EKG, or as an image, such as an X-Ray or a photograph, or a series of images such as a computed tomography (CT) or other scan. Image series data may also have information associated with it such as analysis of anatomy. Lab tests and pathology tests are other types of medical data, as are reports in various formats.

Currently the EMR can collect, store and display a subset of these types of data, such as text and lab tests, but it is not equipped to collect, store, nor display many of the other types of data. More importantly, the EMR is not equipped to analyze and ultimately use the data that it stores. For example, if a patient comes in complaining of chest pain, an EMR system cannot make any assessment of what the cause might be. It cannot even suggest steps to take to determine the cause. The EMR cannot easily display data that may be related to chest pain, such as certain lab tests or reported symptoms. Images, such as a chest X-Ray or a chest CT scan may not even be part of the EMR system and so these important data also cannot be accessed or used via the EMR system.

Even EMR systems, which have the capability of displaying images, do not have the ability to analyze the images for anomalies etc. No current medical data storage or analysis system has the ability to analyze data in different formats and/or from different sources (images, text, reports, voice, etc.). Nor does any current system have the ability to display data in different formats and/or from different sources that is relevant to users' concerns. For example, if a patient comes in complaining of chest pain, no current system can collect and display for the user medical information relating to chest pain, such as symptoms, images, lab tests, current medications, potential medication interactions etc. In addition, no current medical data storage or analysis system has the ability to analyze data in different formats and/or from different sources to create potential next steps or a potential diagnosis/treatment.

### BRIEF DESCRIPTION OF THE DRAWINGS

Embodiments of the invention are illustrated by way of example and not limitation in the figures of the accompanying drawings in which like references indicate similar elements.

FIG. 1 is a block diagram illustrating a medical information system using evolving contextual clinical data technology according one embodiment of the invention.

FIG. 2 is a block diagram illustrating a medical information system according to one embodiment of the invention.

FIG. 3 is a block diagram illustrating an evolving contextual clinical data engine for processing medical information according to one embodiment of the invention.

FIG. 4 is a block diagram illustrating an example of a data collector of a medical information server according to one embodiment of the invention.

FIG. 5 is a flow diagram illustrating a process for processing medical data using evolving contextual clinical data technology according to one embodiment of the invention.

FIG. 6 is a flow diagram illustrating a process for processing medical data using evolving contextual clinical data technology according to one embodiment of the invention.

FIGS. 7A and 7B are screenshots representing graphical user interfaces for providing medical information according to some embodiments of the invention.

FIGS. 8A-8D are screenshots representing graphical user interfaces for providing medical information according to some embodiments of the invention.

FIG. 9 is a flow diagram illustrating a process for processing medical data using evolving contextual clinical data technology according to another embodiment of the invention.

FIGS. 10A and 10B are screenshots representing graphical user interfaces for providing medical information according to some other embodiments of the invention.

FIG. 11 is a flow diagram illustrating a process for processing medical data using evolving contextual clinical data technology according to another embodiment of the invention.

FIGS. 12A-12D are screenshots representing graphical user interfaces for providing medical information according to some other embodiments of the invention.

FIGS. 13A-13B are screenshots representing graphical user interfaces for providing medical information according to some other embodiments of the invention.

FIGS. 14A-14C are screenshots representing graphical user interfaces for providing medical information according to some other embodiments of the invention.

FIGS. 15A and 15B are block diagrams illustrating a cloud-based image processing system according to certain embodiments of the invention.

FIG. 16 is a block diagram of a data processing system, which may be used with one embodiment of the invention.

### DETAILED DESCRIPTION

Various embodiments and aspects of the inventions will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present inventions.

Reference in the specification to “one embodiment” or “an embodiment” means that a particular feature, structure, or characteristic described in conjunction with the embodiment can be included in at least one embodiment of the invention. The appearances of the phrase “in one embodiment” in various places in the specification do not necessarily all refer to the same embodiment.

According to some embodiments, embodiments of the invention provide for the integrating of medical data using evolving contextual clinical data (ECCD) technology, including DICOM (digital imaging and communications in medicine) images, non-DICOM images (such as PNG (Portable Network Graphics), JPEG (joint photographic experts group), GIF (Graphics Interchange Format), BMP (Bitmap), etc.), text, reports, PDF (portable document format) documents, files, audio files, video files, office documents, and other data objects, etc. and display of the relevant data to the end user in a useful way, or “in context.” “In context” means that the data displayed is relevant to whatever the user is searching or viewing. For example, “in context” might mean displaying data relating to a patient ID, accession number, a study ID, login credentials, date, time frame, episode, appointment, body part or area, user, study, insurance code, clinical trial, etc., or any combination of these parameters.

For example, if a user is viewing a particular patient who is complaining of chest pain, the user may click on, or enter, the term “chest pain” and the ECCD system may display data relating to pain and/or heart disease. For example, the system may display an EKG performed recently, or information relating to any changes in EKG data over time. The ECCD system may display lab results relating to heart disease, or changes in lab results over time. The system may display any related symptoms in past reports, visits, dictations, etc., which are related to pain and/or heart disease. The ECCD system may display this information in any appropriate format, including text, images, graphs, charts, spreadsheets etc.

In some embodiments, certain types of data that the ECCD system displays may be manipulated by a user. For example, a user (e.g., a physician, a medical doctor, a professor or researcher, a clinical student, a lab technician, or any other personnel with certain accessing privileges) can refine the data displayed by narrowing or broadening the topic. For example, if the user does not want to see data relating to general pain, but only to heart disease, he/she could narrow the range of data displayed. The user may also want to manipulate the data itself. For example, if the ECCD system displays intelligence indicating that the patient has a 65% stenosis based on past CT or other imaging scan(s), the user is able to drill down into the source of this intelligence and make any changes that he/she sees as necessary (depending on the user's access credentials). In this example, the user would be able to drill down into the actual CT images and the image analysis that was done by the ECCD system to determine if it looks correct and make any corrections. Perhaps the user feels the outline of the stenosis on the images is not quite right
the user can move the outline to improve the analysis. The system will then provide the user with intelligence based on the new stenosis outline, resulting possibly in a higher or lower stenosis number.

In one embodiment, the ECCD system is able to go beyond displaying medical data in context. The ECCD system can also analyze the data to provide intelligence and even diagnoses and recommend next steps/treatment. The analysis can be automatically performed based on a set of rules or published disease management guidelines in view of the medical data without any user intervention or user involvement, or with minimal user involvement. The ECCD system is able to not only analyze image scan series to help interpret them, but is able to combine other medical data from different sources in combination and analyze these data together. The combined medical data can include other medical data (e.g., medical history) of the same patient, medical data of other patients in similar situations, and/or certain predetermined benchmarks associated with that particular medical data item or topic. The ECCD system can provide and display differential diagnoses with probabilities. To narrow down the probabilities, the ECCD system may suggest particular testing to be performed. These types of suggestions may be based on data collected over time and/or over patients, and/or published disease management guidelines.

For example, a patient may have a CT scan performed which shows a slight narrowing of one artery in the heart. This piece of data may not be enough information to help a physician determine whether he/she should treat the stenosis or not. However, this bit of data in combination with other medical data, such as, for example, lab data, symptom data, medical history, family history, medical data from other patients in similar situation with known outcome etc., will provide the physician much more context within which to make his/her decision.

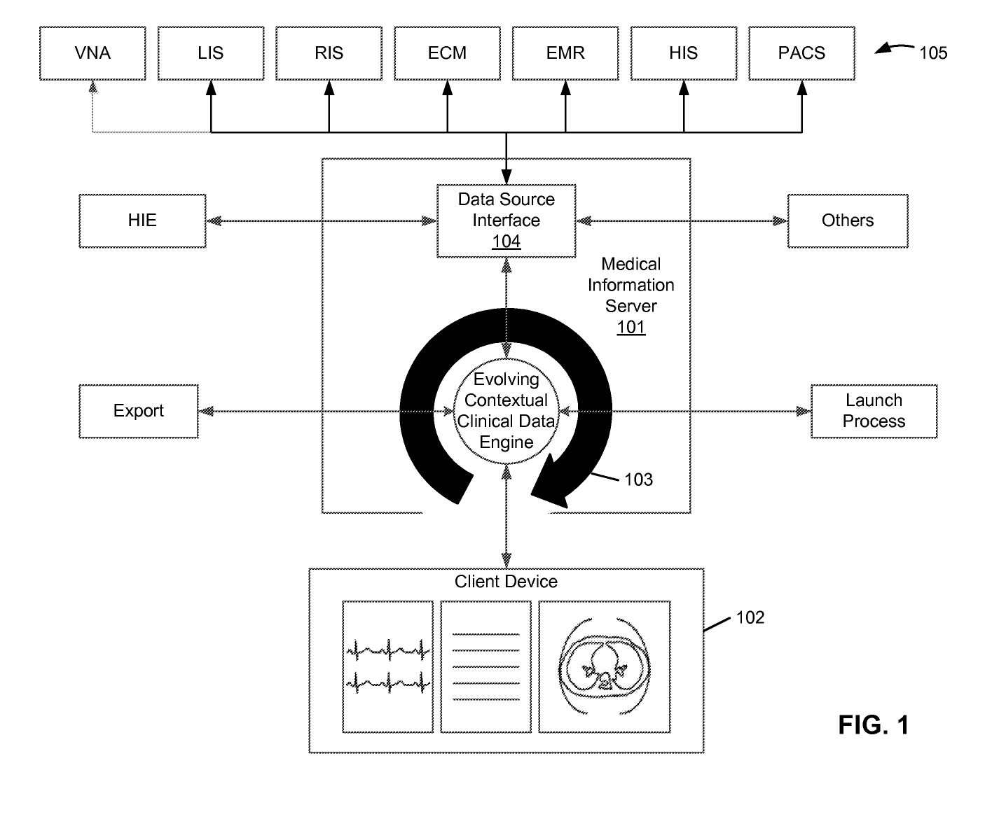

FIG. 1 is a block diagram illustrating a medical information system using Evolving Contextual Clinical Data technology according one embodiment of the invention. Referring to FIG. 1, medical information server 101 is configured to provide medical information to a variety of clients, such as client 102, over a network. In one embodiment, medical information server 101 includes, amongst others, ECCD engine 103 and data source interface 104. ECCD engine 103 may be implemented in a dedicated computing machine having a processor and memory, as well as other hardware components. In one embodiment, ECCD engine 103 may be implemented using an Artificial Intelligence (AI) technology. ECCD engine 103 may also be referred to as an advanced medical data processing engine and interface, or “Eng-Int” for short.

Referring back to FIG. 1, in one embodiment, ECCD Engine 103 performs several functions, including receiving information from a viewer or user, based on a user's input; invoking data collector 104 to query multiple data sources 105 for information relating to the information received from client 102; and integrating and transmitting the data received from the multiple data sources 105 to client 102 to be displayed in the viewer, possibly in different display or viewer areas. This process is repeated in an iterative manner so that the user can narrow or broaden his search, and view the results. This is referred to as an iterative query and is driven by ECCD Engine 103 in response to user interaction with the presented information.

The ECCD engine 103 may determine what types of medical data a user of client 102 is likely interested in receiving based on a set of ECCD rules or models (not shown) that may be modeled based on the prior user interactions or behaviors of a particular user or users. In other words, ECCD is capable of self-learning. ECCD engine 103 communicates, via data source interface or interfaces 104, with data sources 105 to retrieve the medical data (e.g., of a particular patient or patients) based on the recommendation from ECCD engine 103, for example, using a variety of communication protocols over a network that are compatible with the data sources 105. ECCD engine 103 analyzes or mines data objects from several different data sources 105 and integrates the data objects with each other based on common metadata such as patient ID, accession number, date, time frame, body part, body area, medical condition, encounter, procedure, symptom etc.

The ECCD engine 103 uses specific data source interfaces 104 to connect to the various data sources 105. Medical data is pulled from multiple data sources 105, integrated, and transmitted to and displayed at client 102, where client 102 may be a thin client such as a Web browser. In the example as shown in FIG. 1, data sources 105 include Laboratory Information System (LIS), Radiology Information System (RIS), Enterprise Content Management Systems (ECM), Electronic Medical Record (EMR), Hospital Information System (HIS), Picture Archiving and Communication System (PACS), VNA (Vendor Neutral Archive), EMR data, various directories as well as other data sources HIE (health information exchange) servers. However, more or fewer data sources may be applied dependent upon the specific configuration and/or the medical data in demand. Data sources 105 may be managed and/or operated by different organizations or information providers than the organization which operates server 101.

In one embodiment, the medical data provided by data sources 105 may include medical image data in a DICOM format, medical image data in a non-DICOM format, scheduling data, registration data, demographic data, prescription data, billing data, insurance data, dictation data, report data, workflow data, EKG data, best practices reference materials, reference materials, training materials, etc. These data may reside in several locations or systems including HIS, RIS, PACS, LIS, ECM, EMR or other systems. The non-DICOM data may be in several formats including A/V, MPEG, WAV, JPG, PDF, Microsoft Office™ formats and other formats.

Generally, data in the PACS will include DICOM data, where data in the HIS, RIS and LIS, ECM, EMR will include non-DICOM data, including both image and non-image data. HIE data includes data available via a health information exchange system. These data generally include data available across different organizations within a region, community or hospital system, and may be DICOM or non-DICOM data. Other data may include any other relevant data, including data in directories on computers, data from mobile devices, etc.

Since the various systems (e.g., LIS, RIS, ECM, EMR, HIS, PACS, etc.) may use different communication standards, formats, or protocols, such as DICOM, HL7 (health level seven), XDS, HIE, ORU, etc., ECCD engine 103 uses specific connectors or data source interfaces 104 to access the data in the various systems 105. These connectors are generally hidden from the end user at the viewer level, so that the user does not need to worry about these complexities. However, certain users with the appropriate access credentials are able to configure the connectors via the viewer interface, directly with the ECCD engine 103, or through some other interface. Types of data connectors include, but are not limited to, mobile, EMR plugin API (application programming interface), Web services, Web browser uploads/downloads, HL7, directory scanners, DLL (dynamic link library) APIs, XDS (cross-enterprise document sharing), VNA (vendor neutral archive), indexing servers, etc.

The viewer or client 102 in this embodiment may be a thin client, such as a web browser on a computer, a mobile device application on a mobile device, etc. The viewer may or may not require any software or plug-in download/installation. The viewer may have one or more viewer/viewing areas to display the data collected from the various data sources and integrated and received from server 101. The viewing areas may be in frames and/or tabs within a Web browser. The viewing areas may overlap, or be integrated with each other. The viewing areas may be within one another. Preferably the viewer will have more than one viewing area.

The data resulting from the ECCD engine 103's querying may be displayed in the viewer. However, data may also be exported by the ECCD engine 103 for incorporation into a database, report, to integrate with another system etc. The results of the iterative query may also be stored in either the ECCD engine 103 or exported to be stored in another system, or both. The ECCD engine 103 may also launch another process, such as launching another software system, launching a printer to print etc.

In one embodiment, ECCD engine 103 is able to go beyond displaying medical data in context. ECCD engine 103 can also analyze the data to provide intelligence and even diagnoses and recommend next steps/treatment. The analysis can be automatically performed based on a set of rules or established disease management guideline in view of the medical data without any user intervention or user involvement. ECCD engine 103 is able to not only analyze image scan series to help interpret them, but is able to combine other medical data from different sources in combination and analyze these data together. The combined medical data can include other medical data (e.g., medical history, lab data, etc.) of the same patient, medical data of other patients in similar situations, and/or certain predetermined benchmarks associated with that particular medical data item or topic.

According to another embodiment, ECCD engine 103 may provide recommendations based on the analysis of the combined data. Perhaps this patient has had this particular stenosis for years, and all the blood tests/lab tests are normal and the patient is exhibiting no symptoms. This information in combination may indicate to the physician a very different treatment than if the patient had chest pain, changes in blood tests that indicated heart disease or other more concerning data. Without this context, the physician may not be in the best position to make a decision, or may need to repeat tests/scans etc.

In addition to including relevant data for this particular patient, according to one embodiment, ECCD engine 103 can also consider data from other patients. For example, if historically most patients with a stenosis of 65% or over, with chest pains and lab tests in certain ranges, likely went on to have a heart attack within a certain period of time such as one year, ECCD engine 103 may display this information for the physician and ECCD engine 103 may also recommend a treatment related to that particular findings. The aggregate data from other patients/users is sometimes referred to as “ground truth.” The data can be mined and analyzed for any number of things. ECCD engine 103 may analyze treatments performed on patients that fit different profiles and analyze this data along with the outcomes. ECCD engine 103 can then use this data to recommend treatments to other users.

Furthermore, according to one embodiment, in view of the analysis of medical data of a patient, ECCD engine 103 may automatically perform an action or operation based on a set of rules. For example, if data of a particular medical data, issue, or topic exceeds or drops below a predetermined threshold, a notification may be automatically sent to a predetermined recipient, such as the patient and/or a primary care physician, etc.

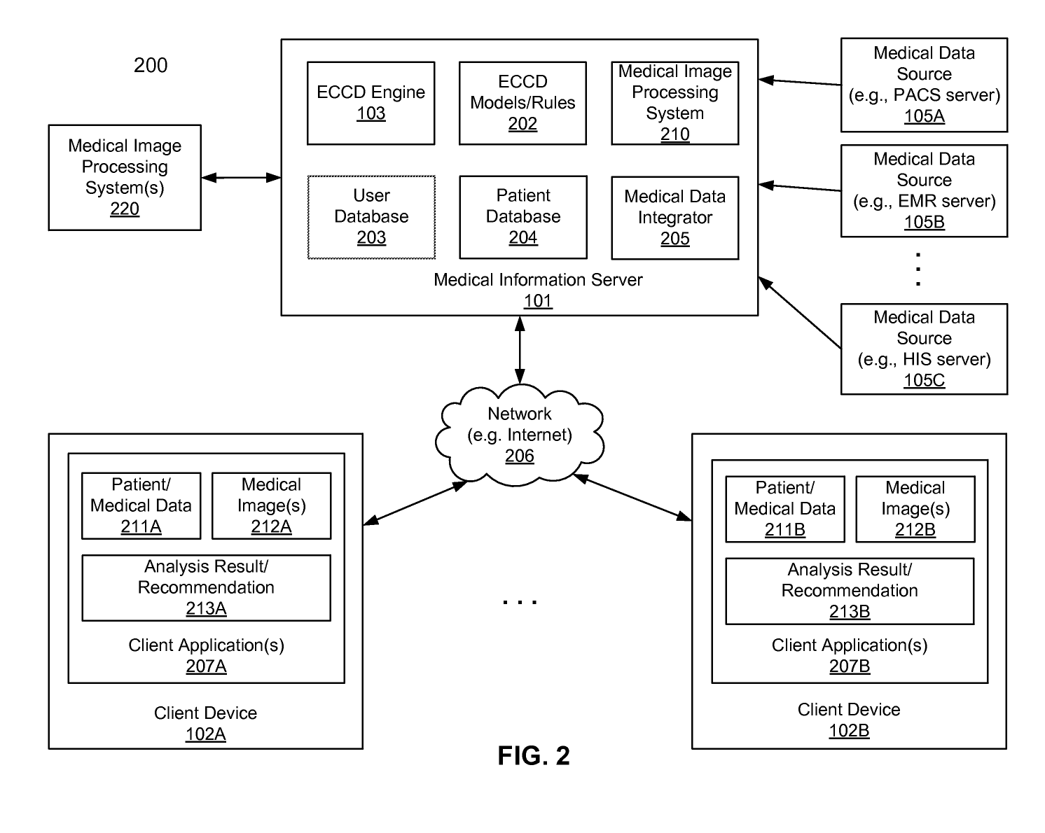

FIG. 2 is a block diagram illustrating a medical information system according to one embodiment of the invention. For example, system 200 may represent the system as shown in FIG. 1, where at least some of the components of server 101, such as ECCD engine 103 and ECCD rules/models 202, may be implemented as part of ECCD engine 103. Referring to FIG. 2, system 200 includes, but is not limited to, one or more clients 102A-102B communicatively coupled to medical information server 101 over Network 206. Network 206 may be a local area network (LAN), a metropolitan area network (MAN), a wide area network (WAN) such as the Internet or an intranet, a private cloud network, a public cloud network, or a combination thereof.

In one embodiment, medical information server 101 hosts therein, amongst others, ECCD engine 103, ECCD models/rules 202, user database(s) 203, patient database(s) 204, medical data integrator 205, and medical image processing system 210. ECCD engine 103 may be implemented using a variety of technologies available to model and generate ECCD rules or models 202 based on user interactions, behaviors, and/or user preferences of a particular user stored in user database 203. The user interactions may be monitored and captured by a user data collector (not shown), which monitors user interactions of client software applications 207-508 at clients 102A-102B. The captured user interactions may then be stored in user database 203 and analyzed by ECCD engine 103 to generate ECCD rules or models 202.

According to one embodiment, when a user of a client, in this example, a user of client 102A, interacts with content (e.g., patient or medical data 211A, medical image 212A) presented at client software 207A (e.g., client application, Web browser), a signal or message representing the user interaction (e.g., click, keystroke, voice command) is transmitted by client application 207A to medical information server 101. The message may include the specific content the user has interacted with, as well as other metadata (e.g., patient ID, body area or body part ID, medical procedure ID, medical appointment, medical condition, user ID, date and time of the interaction, etc.). Based on the message received from client 102A, data integrator 205 invokes ECCD engine 103 and/or ECCD rules/models 202 to determine a set of medical data. The ECCD rules/model 202 may be identified based on a user ID of the user operating client software 207 and/or metadata (e.g., patient ID, body part ID, etc.). Based on the determined data set, data integrator 205 identifies some of the data sources 105A-105C that are able to provide such data. Data integrator 205 communicates with the identified data sources 105A-105C to retrieve the medical data determined or recommended by ECCD engine 103 and/or ECCD rules/models 202, using a variety of communications protocols over a network. Alternatively, data integrator 205 may, based on the message received from client 102A, search some or all available data sources 105A-105C to identify a related set of, and collect the related set of medical data.

Data integrator 205 integrates the retrieved medical data into one or more views of medical information. In one embodiment, data integrator 205 integrates the medical data in a manner that is most appropriate for and/or preferred by the user, for example, based on the ECCD rules/models 202 associated with the user. The one or more views of the medical information are then transmitted from medical information server 101 to client 102A over network 206 and presented to the user by client software application 207A as part of medical data 211A and/or medical images 212A.

Note that the presented medical information may include the medical information requested by the user when the user interacted with the content previously presented at the client. In addition, the medical information may further include information that is not specifically requested by the user, but is determined or recommended by the ECCD engine 103 based on ECCD rules/models 202. For example, when a user requests information concerning a CT scan, based on the ECCD rules or models associated with the user, the ECCD engine may recommend, for example, based on another set of ECCD rules that is associated with the metadata of the CT scan, that one or more lab tests, or an EKG, associated with the CT scan may also be presented to the user. Demographics, family history, clinical notes, etc., and/or a combination of related information may be used to determine the ECCD rules. Although the user did not specifically request the lab tests nor EKG, however, based on the ECCD rules or models, the user may be more likely interested in receiving the lab tests or EKG when viewing the CT scan information or the EKG or lab tests are highly relevant and diagnostically important as determined by ECCD.

Medical images 212A-212B may be rendered or generated by image processing system 210, which may be integrated within medical information server 101 or remotely as part of image processing system 220 hosted by a separate server or cluster of servers over a network. Various data sources 105A-105C may be hosted by one or more servers, which may be operated by the same or different organizations or information providers. In one embodiment, data sources 105A-105C include at least one of LIS, RIS, ECM, EMR, HIS, PACS, VNA and HIE servers. Data integrator 205 communicates with data sources 105A-105C to retrieve medical data using a variety of communication methods or protocols, such as, for example, DICOM, HL7, XDS, HIE, and ORU, etc.

In one embodiment, the medical data of a patient or patients obtained from data sources 105A-105C may be cached or stored in patient database 204. Alternatively, the database may be external, and/or there may be multiple databases. In addition, ECCD engine 103 may perform an analysis on the collected patient medical data of a patient in question stored in database 204. For example, based on the current medical data that is interested by a user of a client device, ECCD engine 103 may determine a likelihood of an abnormal medical condition or disease associated with the medical data, issue, or topic associated with the patient may occur for the patient. The determination of such a likelihood of the medical condition may be determined based on the analysis result in view of the patient's medical history or medical records in the past or certain related benchmarks or thresholds, and/or other patients' data.

In one embodiment, ECCD engine 103 may invoke a medical image processing system to perform an image processing operation on an image of a body part of the patient to produce certain derived image quantitative data or measurement data. The image may be selected by the user of the client device as part of medical data obtained from data sources 105A-105C. The image quantitative data may be used to determine or measure the size and/or shape of a particular body part of the medical image. The image quantitative data may be compared with a corresponding benchmark associated with the type of the image to determine whether a particular medical condition, medical issue, or disease may likely occur in near future or a specified time frame. The likelihood of such occurrence may further be predicted or determined based on a trend of same type of medical data of the patient as part of medical history of the patient and/or other patients' data.

In one embodiment, ECCD engine 103 may further recommend an action or course of action to be taken in view of the analysis. ECCD engine 103 may further automatically perform a predetermined action (e.g., sending a notification to a preconfigured recipient) based on the analysis. The analysis result and the recommended course of action may be transmitted to client devices 102A-102B as part of analysis result and recommendations 213A-213B, respectively. The analysis result may further include information indicating that the predetermined action has been performed and/or the recommendation. All of these operations may be performed automatically without user intervention based on a set of rules in view of the analysis.

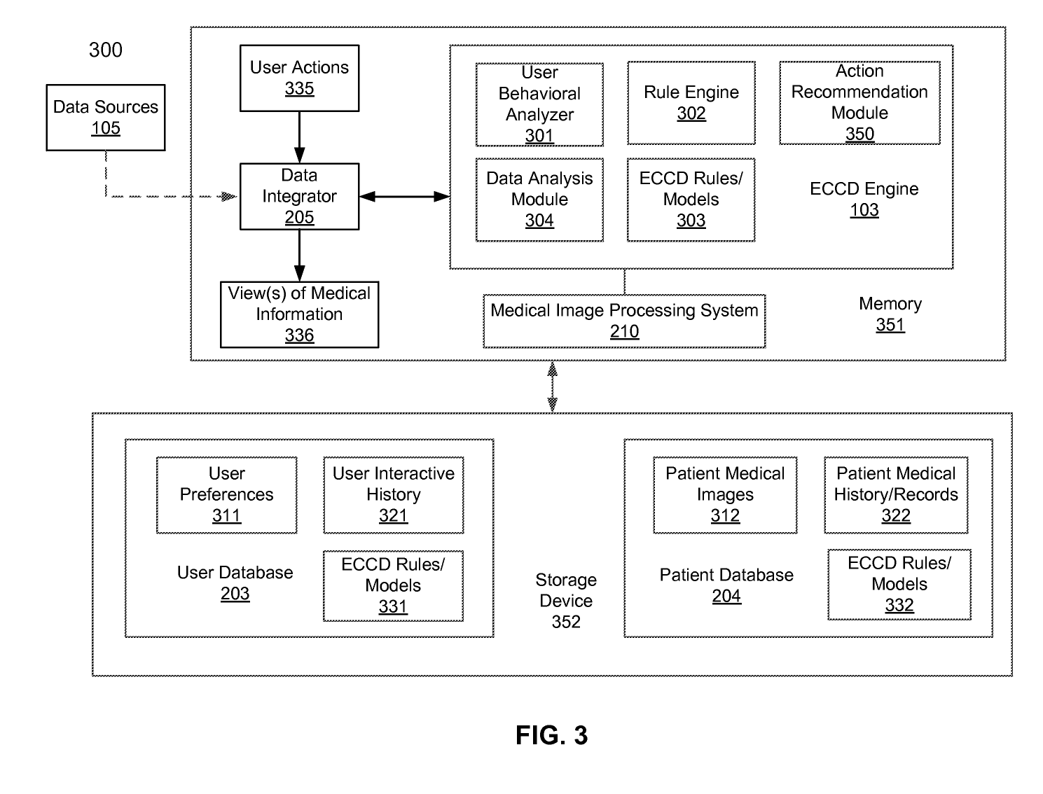

FIG. 3 is a block diagram illustrating an evolving contextual clinical data engine for processing medical information according to one embodiment of the invention. Referring to FIG. 3, system 300 may be implemented as part of medical information server 101 of FIG. 1. In one embodiment, ECCD engine 103 includes, but is not limited to, user behavioral analyzer 301, rule engine 302, and ECCD rules or models 303 generated by ECCD engine 302, which may be loaded in memory 351 and executed by one or more processors (not shown). In one embodiment, user behavioral analyzer 301 analyzes user interactive data of a user to determine user behavioral patterns with respect to accessing different medical information of a patient. For example, user behavioral analyzer 301 may access user database 203 of different users to analyze user interaction history 321 of the corresponding users to determine their respective behavioral patterns. The user interaction history 321 may be collected by user data collector 204 of FIG. 2 and stored in the user database 203, which may be implemented in one or more databases in a persistent storage device 352 such as hard drives. The user behavioral patterns may be determined or modeled using a variety of algorithms or technologies 304, as described above. User behavioral patterns may be used for that individual user or aggregated across more than one user.

Based on the user behavioral patterns determined by user behavioral analyzer 301, ECCD rule engine 302 compiles and generates a set of ECCD rules or models 303 for each of the users based on their respective behaviors and personal preferences 311. The ECCD rules and/or models are then stored in the corresponding user databases as part of ECCD rules/models 331 or alternatively, they may be centrally maintained by ECCD engine 103 as part of ECCD rules/models 303. Subsequently, when a new user action 335 is received for accessing certain medical data, data integrator 205 invokes ECCD engine 103 to access ECCD rules/models 303 and/or ECCD rules/models 331 corresponding to the user to determine additional medical data that the user is likely interested in receiving. Data integrator 205 then communicates with the associated data sources 105 to retrieve the requested medical data (e.g., first medical data) and the additional medical data (e.g., second medical data). The retrieved medical data is then integrated by data integrator 205 to generate one or more views of medical information 336. The one or more views of medical information 336 is then transmitted to a client device of the user to be presented therein.

Furthermore, the user action 335 may be captured and stored in persistent device 352 as part of user interaction history 321. The updated user interaction history may be used or “learned” by ECCD engine 103 to train or adjust the corresponding ECCD rules or models for future use. For example, if users rarely or never request EKG data when viewing scans of the brain, then the ECCD engine 103 will learn, by configuring the corresponding ECCD rules or models, not to present the EKG data when a user is viewing a scan of the brain. In this situation, the user may need to make a specific query to view the EKG information if he/she wants to, and such a user action may trigger further modification of its ECCD rules or models.

In another example, based on the past user behaviors, a user may always want to view images representing an EKG of a patient when he is viewing a CT scan of the chest area, as a cardiologist might. Once the user has made this request one or more times, the ECCD engine 103 can learn that this particular user frequently desires viewing these images at the same time, and ECCD engine 103 may modify the ECCD rules/models to present the EKG data when the user subsequently requests the viewing of a CT scan of the chest area. If many other users also frequently request the EKG data while viewing CT scans of the chest area, the ECCD engine 103 can interpret this data and raise the likelihood of presenting the EKG data when chest CT scan data is requested for all users, or a subset of all users, for example, cardiologists. In this way, the ECCD engine 103 is able to “learn” what “in context” means for different users or different types of users or all users.

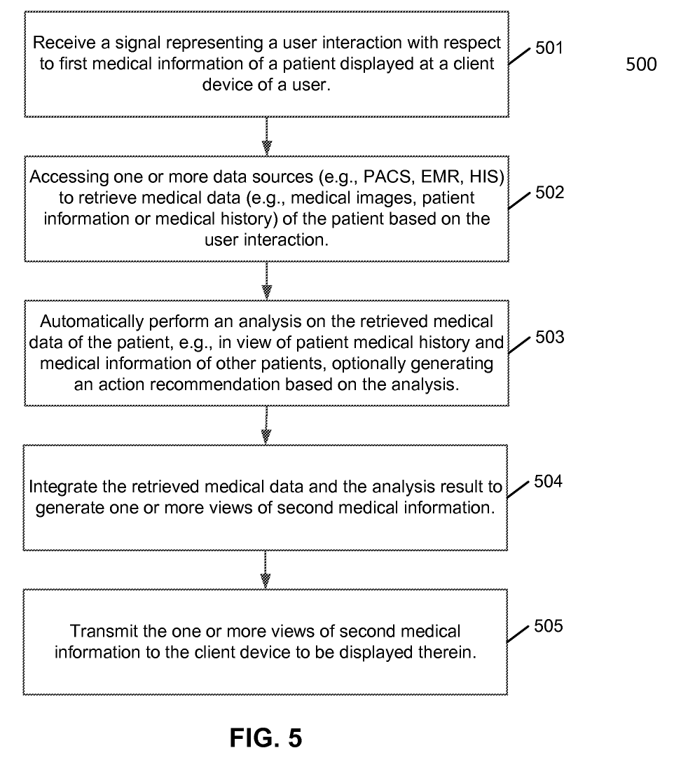

Another way that the ECCD engine 103 may learn from its users is by tracking click through rates and view times (e.g., monitored by user data collector 204 of FIG. 5) and by connecting this information within the ECCD engine 103 with the user, type of study, individual study, type of information, other information being displayed, etc. A shorter click through rate and/or a shorter viewing time may indicate less relevant information while a longer click through rate and/or a longer viewing time may indicate more relevant information. Using this collected information over time, the ECCD engine 103 grows more intelligent as it is used by refining the content it presents. For example, a user or users may view lab data which is older than 3 months for only a few seconds, but view newer lab data for much longer, indicating that the older information is not very useful. Using this information, the ECCD engine 103 may, in this example, become less likely to present lab data more than three months old to a user or users.

Another example of how the ECCD engine 103 may learn from its users is by tracking total interpretation time. Total interpretation time may be the time to view multiple related images or information objects, and/or it may be the time to perform certain advanced image processing steps, and/or may be the total time reviewing information relating to a single patient. This information can be analyzed by the ECCD engine 103 to determine trends in reading times. This information can ultimately be used to reduce total reading times. For example, if the ECCD engine 103 determines that a physician spends 30 minutes reviewing a particular patient when part of the review process includes viewing a CT scan by itself, but only 10 minutes reviewing a patient when a CT scan is presented next to a prior CT scan, the system may increase the likelihood that it will display older or newer CT scans when the user chooses a CT scan to view.

Another example of how the ECCD engine 103 may learn from its users is by directly requesting user feedback. For example, the ECCD engine 103 may ask if displaying certain data objects has been useful or not useful. By collecting and analyzing this information, the ECCD engine 103 can more quickly learn what data objects are more or less relevant. The ECCD engine 103 also knows the combination of data objects it is displaying at any given time and can incorporate this knowledge into the analysis. For example, if users indicate that an EKG is not very useful when displayed by itself, or in conjunction with a colonoscopy, but indicate that an EKG is very useful when displayed along aide a CT scan of a heart, the ECCD engine 103 will learn to display the EKG more often when a CT scan of a heart is being viewed, and possibly less often otherwise. The above examples are just few of possible situations, which may be represented by ECCD rules/models 303 and 331. Other possibilities can also be applied.

According to one embodiment, the patient medical data of a particular patient or patients may be obtained from data sources 105 and cached and stored in patient database 204 maintained in storage device 352. The information stored in patient database 204 may include, but it is not limited to, patient medical images 312, patient medical history or records 322, and an optional set of ECCD rules 332 governing how to process the information. The patient database may further store other patients' medical data, as well as certain medical data benchmarks (not shown). Alternatively, the patient medical data may not be stored/cached, and instead, the data is accessed in real time from data sources 105.

According to one embodiment, ECCD engine 103 further include an analysis module 304 and an action recommendation module 350. Analysis module 304 may cause the medical information stored in patient database 204 to be loaded into memory 351 and perform an analysis on the medical information of a particular patient. For example, analysis module 304 may invoke image processing system 210 to process medical images 312 to provide quantitative data concerning the images (e.g., measurements regarding the size and/or shape of a body part from the images). Analysis module 304 then performs an analysis on the image quantitative data to determine whether the patient is likely to have a certain medical condition occurred in the near future or a specified time frame. The determination may be performed in view patient's medical history 322 and/or other patients' similar medical data. The determination may be automatically performed based on a set of ECCD rules 332, which may be configured based on the type of medical data and/or the associated patient(s). Alternatively, the patient medical data may not be stored/cached, and instead, the data is accessed in real time from data sources 105.

According to another embodiment, based on the medical data and the corresponding analysis thereof, recommendation module 350 is configured to determine one or more recommendations of course of actions to be taken. The recommendation may be determined based on ECCD rules 303 in view of the medical data and/or the analysis thereof. The analysis result and the recommendation may be incorporated into view(s) of medical information 336 by data integrator 205, which may be transmitted to a client device of a user over a network to be displayed therein.

Note that some or all of the components as shown and described above (e.g., ECCD engine 103, image processing system 210) may be implemented in software, hardware, or a combination thereof. For example, such components can be implemented as software installed and stored in a persistent storage device, which can be loaded and executed in a memory by a processor (not shown) to carry out the processes or operations described throughout this application. Alternatively, such components can be implemented as executable code programmed or embedded into dedicated hardware such as an integrated circuit (e.g., an application specific IC or ASIC), a GPU (Graphics Processing Unit), a digital signal processor (DSP), or a field programmable gate array (FPGA), which can be accessed via a corresponding driver and/or operating system from an application. Furthermore, such components can be implemented as specific hardware logic in a processor or processor core as part of an instruction set accessible by a software component via one or more specific instructions.

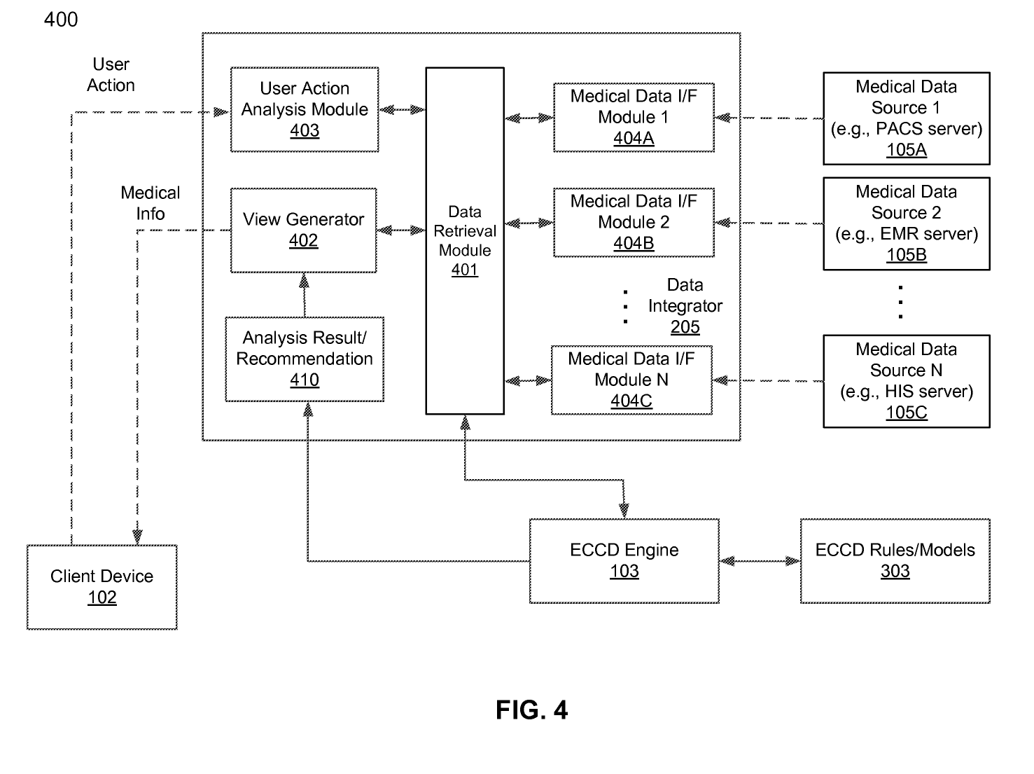

FIG. 4 is a block diagram illustrating an example of a data collector of a medical information server according to one embodiment of the invention. Referring to FIG. 4, data integrator 205 includes, but is not limited to, data retrieval module 401, view generator 402, user action analysis module 403, and medical data interface (I/F) modules 404A-404C. According to one embodiment, when a user interacts with medical content presented by client device 102, a signal or message is transmitted from client device 102 to medical information server 101 over a network and such signal or message is received by user action analysis module 403. The received message may include certain metadata indicating what content item the user interacted with and other identifying information (e.g., user ID, patient ID, medical procedure ID, body area or body part ID, date and/or time of the interaction, medical condition ID, medical appointment ID, etc.) User action analysis module 403 extracts the information from the message and performs an analysis on the extracted information.

In one embodiment, based on the analysis, user action analysis module 403 invokes ECCD engine 103 by providing the information of the results of analysis and/or the extracted metadata from the message. In response, ECCD engine 103 identifies a set of ECCD rules or models from ECCD rules/models 303 that is associated with the user of client device 102, for example, based on a user ID of the user. ECCD engine 103 then derives a set of one or more actions or recommendations based on the inputs provided by user action analysis module 403 using the corresponding ECCD rules or models associated with the user. The recommendations may include collecting or displaying certain additional medical data that is related to the medical data requested by the user, where the additional medical data may be envisioned or predicted by ECCD engine 103 that the user is likely interested in receiving in view of the requested medical data and/or the user action extracted from the message received from client device 102.

Based on the first medical data originally requested by the user and the second medical data additionally recommended by ECCD engine 103, data retrieval module 401 identifies one or more of data sources 105A-105C that provide such medical data. For example, data retrieval module 401 may determine the identifiers for different medical data requested by the user and those recommended by ECCD engine 103. Data retrieve module 401 may maintain and/or access a database or data structure that maps the medical data identifiers to the corresponding data sources. Based on the identified data sources, data retrieval module 401 invokes or calls corresponding medical data interface modules 404A-404C, which in turn access the data sources 105A-105C, respectively.

Medical data interface modules 404A-404C include interface logic (either in software, hardware, or a combination of both) that is specifically designed to handle communications with a specific one of data sources 105A-105C. Each of data interface modules 404A-404C includes functionalities that are specifically configured to communicate with a corresponding one or more of data sources 105A-105C, using specific communication protocols (e.g., TCP/IP, DICOM, HL7, XDS, HIE, ORU, etc.) or application programming interfaces (APIs) that are compatible with or recognized by the corresponding data source. This includes proper protocol signaling or calling convention, handshaking, data exchange, and authentication of different users using associated authentication credentials.

According to one embodiment, data interface modules 404A-404C may also reformat the medical data (e.g., raw data) received from data sources 105A-105C to a format common to or expected by data retrieval module 401 or alternatively, data retrieval module 401 may handle the reformatting operation. In one embodiment, each of data interface modules 404A-404C includes a plugin interface module for the corresponding data source. Data interface modules 404A-404C can handle different types of data, such as, for example, DICOM images, non-DICOM images, text, reports, PDF documents, JPEG files, audio files, video files, office documents, and other data objects. Data sources 105A-105C may be hosted by a variety of servers or clusters of servers, including LIS, RIS, ECM, EMR, HIS, PACS, and/or HIE servers.

Once the medical data has been retrieved by data retrieval module 401 from data sources 105A-105C, the medical data is integrated by view generator 402 to generate one or more views of medical information. The one or more views of medical information may be arranged according to a layout preferred by the user based on the user preferences. Alternatively, views of medical information may be formulated in a manner recommended by ECCD engine 103 based on the ECCD rules or models 303, and/or further in view of the user preferences. For example, less frequently used image processing tools may not be presented to the user or presented at a lower priority with respect to other more frequently used image processing tools, etc. The one or more views of medical information are then transmitted to client device 102 to be presented therein. If the user further interacts with the medical information, the user interaction is captured again and sent to data integrator 205, and the above processes are iteratively performed.

According to one embodiment, ECCD engine 103 is communicatively coupled to data retrieval module 401 to access the retrieved data, which may be stored in a database of a persistent storage device (e.g., database 204), or may be accessed directly from data sources 105. ECCD engine 103 performs an analysis on the medical data stored therein, determines a probability of an abnormal medical condition or disease, generates one or more recommendations, and optionally performs a predetermined action based on ECCD rules 303 as described above. The analysis result and recommendation 410 generated by ECCD engine 103 may be integrated into a view of medical information by view generator 402 and transmitted to client device 102.

FIG. 5 is a flow diagram illustrating a process for processing medical data using evolving contextual clinical data technology according to one embodiment of the invention. Process 500 may be performed by processing logic, which may include software, hardware, or a combination thereof. For example, process 500 may be performed by medical information server 101. Referring to FIG. 5, at block 501, processing logic receives a signal or message presenting a user interaction with respect to medical information (e.g., first medical information) displayed at a client device of a user. At block 502, processing logic determines the types of medical data requested and communicates with one or more data sources (e.g., PACS, EMR, HIS) to retrieve medical data (e.g., medical images, patient information or medical records) of the patient based on the user interaction. At block 503, processing logic automatically performs an analysis on the retrieved medical data of the patient, e.g., in view of patient medical history and/or other patients' medical data. Processing logic optionally generates a recommendation of course of action based on the analysis. At block 504, processing logic integrates the received medical data and the analysis result to generate one or more views of medical information. At block 505, the views of medical information are then transmitted to the client device to be displayed therein.

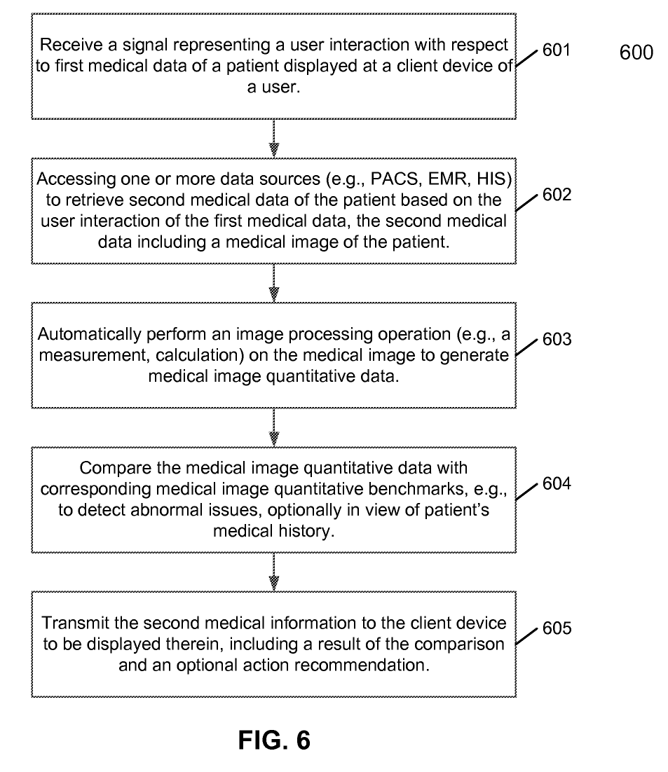

FIG. 6 is a flow diagram illustrating a process for processing medical data using evolving contextual clinical data technology according to one embodiment of the invention. Process 600 may be performed by processing logic, which may include software, hardware, or a combination thereof. For example, process 600 may be performed by medical information server 101. Referring to FIG. 6, at block 601, processing logic receives a signal or message presenting a user interaction with respect to medical information (e.g., first medical information) displayed at a client device of a user. At block 602, processing logic determines the types of medical data requested and communicates with one or more data sources (e.g., PACS, EMR, HIS) to retrieve medical data (e.g., medical images, patient information or medical records) of the patient based on the user interaction. At block 603, processing logic automatically performs an image processing operation (e.g., measurement and/or calculation) on a medical image to generate medical image quantitative data. At block 604, processing logic compares the medical image quantitative data with the corresponding benchmarks to detect any abnormal medical condition or issue, optionally in view of patient's medical history. At block 605, the retrieved medical information and the result of the comparison of the image quantitative data are transmitted to the client device to be displayed therein.

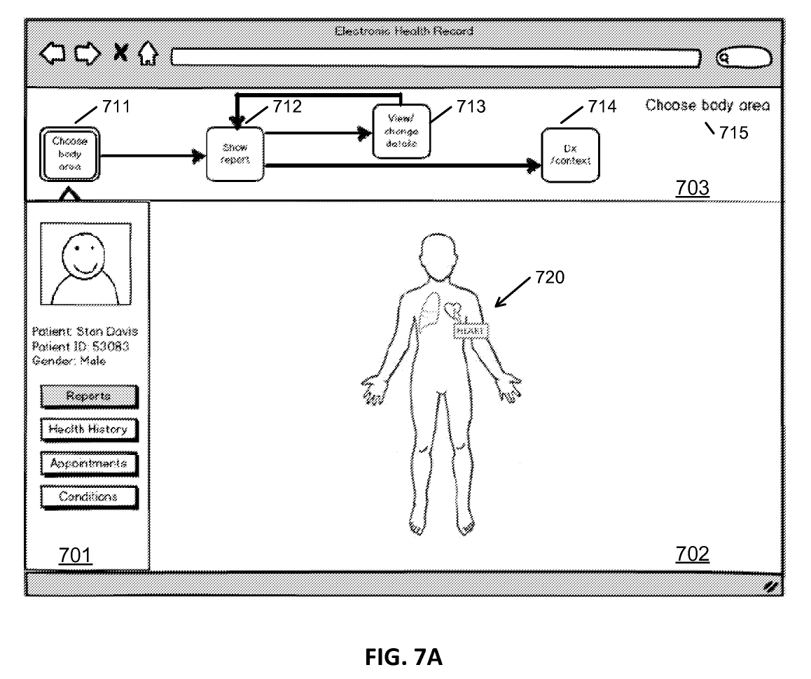

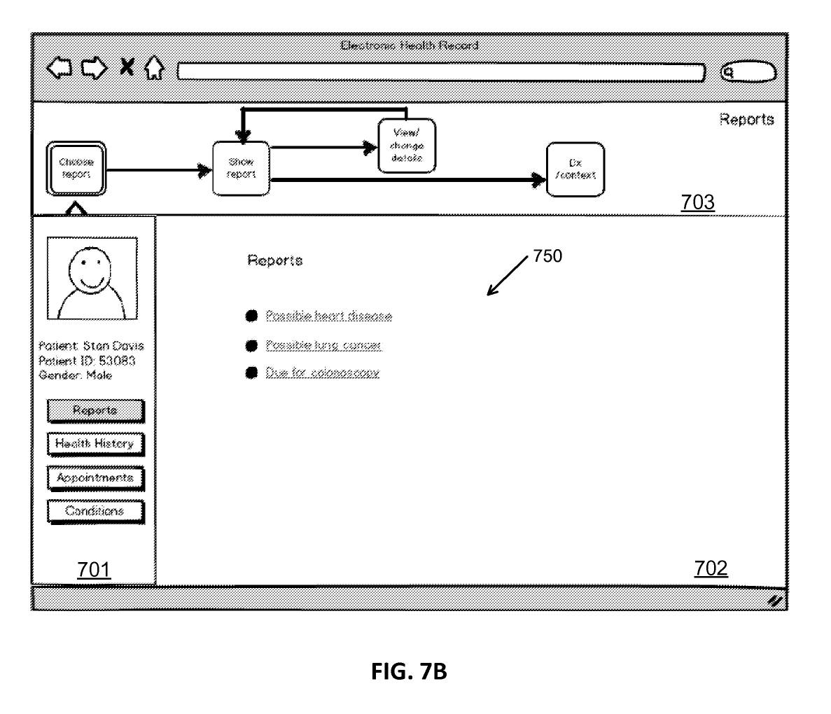

FIGS. 7A and 7B are screenshots representing graphical user interfaces for providing medical information according to some embodiments of the invention. For example, the graphical user interface (GUI) pages as shown in FIGS. 7A-7B may be generated by medical information server 101, transmitted from medical information server 101 to client devices 102A-102B over network 206, and presented by client applications 207A-207B at client devices 102A-102B as shown in FIG. 2. User interactions with the GUI pages are captured and transmitted from the client device to medical information server 101. The user interactions are then interpreted or analyzed by medical information server 101 and in response to the user interaction, medical information server 101 performs proper actions, such as, image processing operations, information retrieval operations, and data processing and/or integration operations, analysis, recommendation, and returns processing results back to the client. The processing results are presented and/or integrated with the existing information at a display device of the client.

Referring to FIG. 7A, in this embodiment, the GUI as shown may represent an electronic medical record (EMR) or electronic health record (EHR) viewer interface, which may be indicated in the title area of the GUI. In one embodiment, the GUI page includes a first display area 701 to display patient identifying information, a second display area 702 to display detailed information about the patient, and a third display area 703 to display a processing timeline of one or more processing stages (e.g., a workflow with one or more workflow stages). The GUI shows one way to access medical data that is associated with a particular patient. In this example, a user can access medical data via body areas, such as a head, a lung, a heart, etc. of a graphical representation of a human body.

In this example, the viewer/client is shown as a web browser. Across the top of the browser window in display area 703 is a timeline, or process line, or workflow, of the data viewing/processing process. This workflow diagram shows the user where he/she is in the process. This GUI indicates that the user is in the “choose body area” stage 711 of the process. In this sample screen, this is communicated by darkened outline or highlighting icon 711 and arrow indicator underneath. Page title 715 shows the user where within the workflow process he/she is, in this case, in the “Choose body area” step. A representation of a human body is show via body 720. A cursor or pointer can be used with body 720 to indicate different body areas, such as the heart, lungs, chest, head, abdomen, intestines etc. In this figure, cursor is shown hovering over the heart area which causes popup text as a hint to become visible. In this way the user can move the cursor around the body and determine which area of the body he/she wants to click.

Display area 701 displays patient information such as a patient name, a patient ID, and gender, etc. Display area 701 further includes one or more navigation buttons or controls to access different areas of the HER associated with the selected patient, such as, for example, medical reports of the patient, patient's medical history, make or view a medical appointment with a physician, and view certain medical conditions of the patient. Dependent upon the accessing privileges of the user (e.g., a doctor, a lap technician, a clinical student, a professor, etc.), some of the information may or may not be accessible and if it is accessible, some sensitive information may be redacted or invisible.

FIG. 7B shows an alternative presentation to FIG. 7A. In FIG. 7A, a user was presented with a body diagram and chose his/her report based on body part. In FIG. 7B, the user is presented with a number of possible ailments/diseases/conditions which the ECCD system has presented based on its analysis of the patient's (and possibly other patients') data. In this example, patient X, which is identified in display area 701, has three areas of concern: possible heart disease, possible lung cancer, and an overdue colonoscopy. These are displayed in list 750. An activation on any of these links would bring the user to screens with more information associated with the selected link.

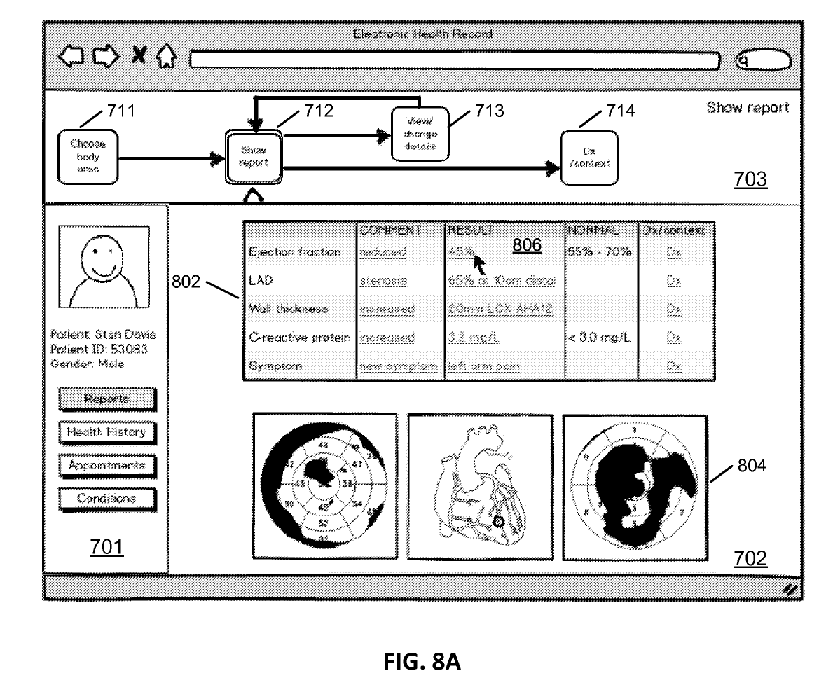

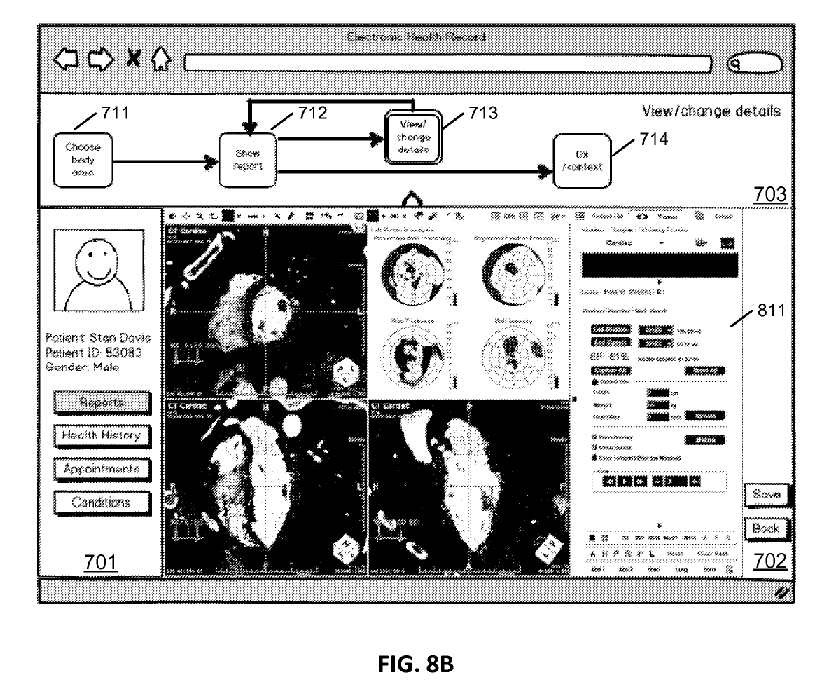

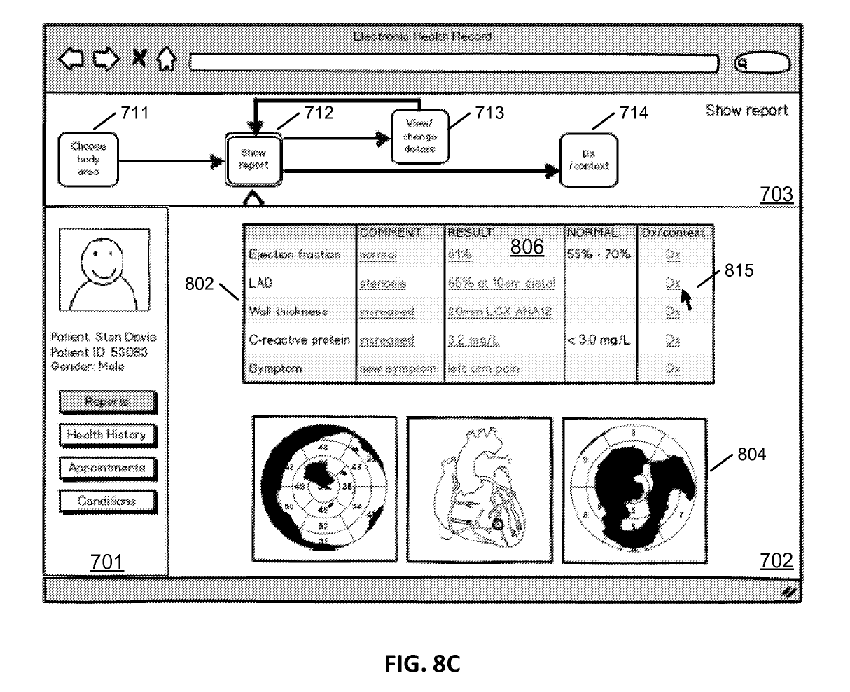

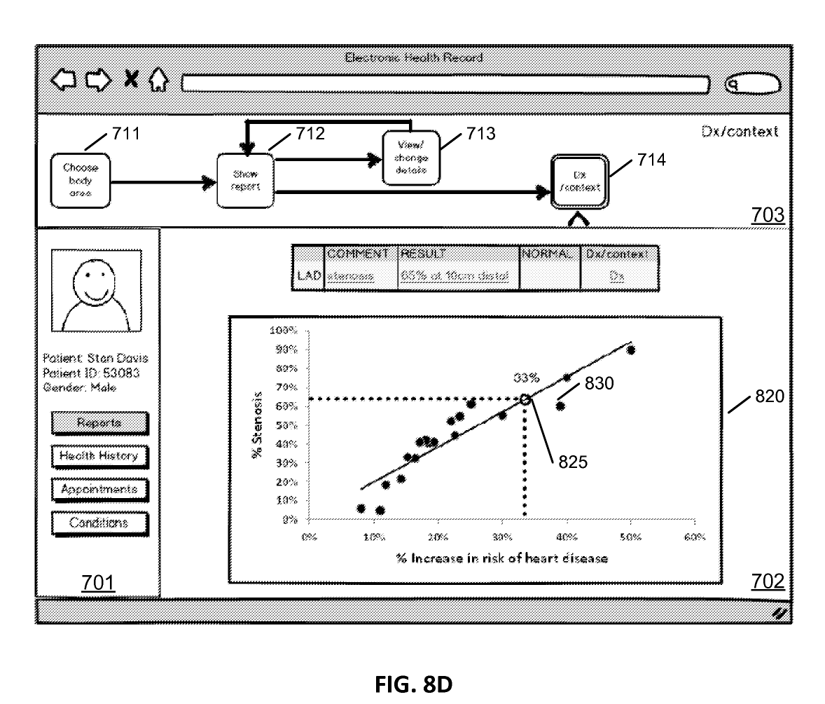

FIGS. 8A-8D are screenshots representing graphical user interfaces for providing medical information according to some embodiments of the invention. For example, FIGS. 8A-8D may be generated and displayed in response to a user interaction with FIGS. 7A-7B. Referring to FIG. 8A, in this example, the GUI page is generated and displayed in response to a user interaction of selecting a heart from a human body representation of FIG. 7A. In response to a user selection of a heart from a client application of a client device (e.g., Web browser), the client application sends a request to medical information server 101 to request the medical information associated with the heart of the corresponding patient. The request may include a patient ID identifying the patient, a body part ID identifying the heart, and other necessary information (e.g., user ID identifying the user to determine the accessing privileges).

In response to the request, server 101 is configured to determine any related medical data of the patient, in this example, medical data related to the heart, as well as other data that the user may be interested in receiving as described above. Server 101 then communicates with one or more medical data servers of different information providers to obtain the related medical data of the patient. In addition, an analysis may be automatically performed and a recommendation of a course of action may be generated based on the analysis. Further, a predetermined action (e.g., sending a notification to a predetermined recipient) may also be automatically performed based on a set of rules that is associated with the type of medical data, the user, and/or the patient, as described above. Server 101 then integrates the medical data and the analysis result to generate one or more views of medical information and transmits the views of medical information back to the client device to be displayed as part of GUI pages as shown in FIGS. 8A-8D.

Referring back to FIG. 8A, the GUI page shows data relating to the heart of this patient. In this example, the data is shown in table 802 as well as images 804 that are related to the heart of the patient, in response to an activation or selection of a heart from FIG. 7A. The processing stage timeline in display area 703 also indicates, via icon 712, that the current processing stage is a show-report stage. In this example, the medical data received from the medical information server 101 includes table 802 having various types of medical data and medical images 804. The data in table 802 and images 804 are generated by the ECCD system and transmitted to a client device of the user. In this example, the data is displayed within a frame, tab, or area of a Web browser, although the data may be displayed in a separate window or pop-up window. The data displayed may be data which is stored in various different data sources, such as the RIS, PACS, LIS, etc. In this example, the medical data presented includes various types of data items including ejection fraction, LAD, wall thickness, C-reactive protein, and symptom.

As described above, the ECCD system may also have performed further analysis on the data. For example, in table 802 ejection fraction data 806 is shown as 45%. This number may be determined by the ECCD system by analyzing image series data, for example, CT scan image series data. Also, comparing results to normal ranges may be part of the analysis and display. The data included in this sample table include data from images, image analysis, lab reports, and symptoms. These data generally reside in very different server systems and databases. The ECCD system is able to associate the relevant data with this patient, and this body part, the heart, perform any relevant analyses, and present the relevant data to the user in the viewer in context. The viewer may be stand-alone, or may be integrated with another software system such as an EHR software system or clinical trials software system.

In this example, each data item includes a comment section having automatically generated comment such as a trend of the data (e.g., increase, decrease, potential new symptom, a possible abnormal medical condition or disease, etc.), which may be determined based on the patient's medical history and/or in view of other patients' similar data. Each data item further includes a result section displaying the analysis or computing result and a normal range of the data item, as well as a link (e.g., “Dx/Context”) to access further detailed information of the data item.

The ECCD system may incorporate several data processing/analysis tools, some of which are described further below. Other types of data processing/analysis include one or more of the following: comparing data to normal ranges, diseased ranges, comparing data to those of others in a patient database, combining data to use in analysis and/or comparison, comparing data to “ground truth” or other standard, analyzing data over time, analyzing changes in data, analyzing data to determine best treatment path, analyzing data to determine what data is missing and should be collected, analyzing data to determine disease risks, analyzing data to determine possible side effects of treatments, analyzing data to determine efficacy of treatments, and analyzing data from multiple sources and in multiple formats.

The types of data analyzed may include one or more of the following: image data, processed image data (for example as performed by the tools listed toward the end of this document), lab test data, pathology data, symptoms data, physician/technician comments/observations/dictation, medication data, treatment data, results data, report data, patient demographic data, patient medical history data, genetic data, genome data, trend data, aggregated patient data, ground truth data (data driven or expert driven), standards data, research data, and clinical trial data.

Since the ECCD system can gather medical data from a variety of sources in a variety of formats and/or communications protocols, it can perform an analysis on the data in any number of ways. The ECCD system can also generate the data and/or analyses in context, whether that context is a patient context, a body part context, disease context, treatment context, clinical or research study context or any other context. In this way the ECCD system is truly an intelligent system, without or with less user intervention.

For example, the ECCD system may access and analyze image and other data connected with patient X. when a user is reviewing patient X's EMR, the user may want to review information associated with the heart so the user clicks on the heart, for example as shown in FIG. 7A. Note that although selecting or activating on the heart may cause the ECCD system to collect and/or analyze contextual data in real time, some or all of the data collection and/or analysis may also be performed automatically and in the background by the ECCD system. Which data collection and/or analysis is done in real time vs. which is done beforehand may depend on the resources necessary to collect/process/analyze the data as well as the frequency with which the data is changed and/or updated.

In this example, the ECCD system analyzes CT scan data and determines that patient X has a 65% stenosis of the LAD (left anterior descending) artery of the heart. The ECCD system then compiles this data in the viewer. The ECCD system may also access and present lab data relating to the heart, as well as symptom data and physician report/dictation/notes data which relates to the heart. For example, the ECCD system may determine that the C-reactive protein level is slightly increased. The ECCD system may also have detected the symptom “left arm pain” in recent physician notes, but not in previous notes/symptoms/reports, and display that “left arm pain” is a new symptom. The ECCD system may also analyze heart imaging data to determine that the heart wall thickness is increased, and that the ejection fraction of the heart is below normal by comparing the ejection fraction result to a standard. Patient X may also have data (for example, images, symptom, treatment etc. data) relating to a broken arm 2 years ago. Although the ECCD system has access to all these data, these data are not displayed when the user is viewing data relating to patient X's heart, because the ECCD system has determined that these broken arm related data are less relevant to the heart.

FIG. 8A shows an example of how the ECCD system may display the information it has produced. Note that several of the data objects are underlined and therefor “clickable” or linked to more information. The user may or may not want to dig deeper into the underlying data, but if he/she wants to view, refine or even alter the data, these links would allow him/her to do so. In this example, the comments, results and Dx (diagnosis)/context data objects are linked. The images may be linked also. If the user clicks on one of the comments, he/she may be brought to a screen showing the full comment with the linked word in context. Or, if the comment is a result of data analysis, the data and/or algorithm may be displayed when a user clicks the link. If the user clicks on one of the result links, the ECCD system will display a screen that shows how the result was determined.

For example, if the user clicks on the ejection fraction data 806, which is also linked, the ECCD system may compiles and cause the client device to display another GUI page to display the corresponding detailed information similar to the GUI page as shown FIG. 8B. Referring to FIG. 8B, which is in view/change detail stage 713, the viewing area/tab/frame toward the lower right area of the browser or client now shows some advanced image processing views and tools. In this example, the ECCD system generates and causes the client device to display the method and data it has used to calculate the ejection fraction of the heart from heart scan images. The right side of display area 702 includes image processing tools. The left side of display area 702 on displays scan images along with segmentation lines and charts. Segmentation lines are the lines representing the borders around and within the heart tissue that the ECCD system has analyzed. In the example of ejection fraction, the ECCD system determines the volume of the chambers of the heart and draws outlines at the inner surface of the chambers. These outlines are also tracked over time, as the heart beats. The calculated ejection fraction is calculated and displayed by the ECCD system. This figure shows only one image in 3 of the 4 images areas on the left, but these images may represent image series, so that the user can scroll through hundred, thousands or more images of one or more scans.

Not only does the ECCD system display detailed image data and analysis information as shown in FIG. 8B, but the ECCD system also allows the user (depending on login credentials) to make changes to the data. For example, if the user believes the outlines that the ECCD system has displayed around the heart should be drawn differently, the user can use a mouse or other pointer to move the outlines to better reflect the anatomy of the heart. The user interaction is then transmitted from the client device to the ECCD server. Based on the user interaction, the ECCD system can then recalculate the ejection fraction (or other parameter) based on the changed data. This new analysis can then be used in the overall patient analysis.

If the user has made changes, he/she may click the “save” button to save the data and have it incorporated into the overall analysis, which may be transmitted back to the ECCD server for update. Alternatively, the user may click the “back” button to go back to the previous screen and disregard the changes.

FIG. 8C shows a similar GUI page as shown in FIG. 8A, however the ejection fraction has changed as a result of the user changing the parameters used in this calculation. This may have been done by the user changing the outlines of the chambers of the heart as was shown in FIG. 8B, which is now back to stage 712.

The ECCD system can also analyze data to suggest possible diagnoses or treatment paths for a patient. And/or, the ECCD system can display the data relating to the current patient in the context of other patients or in the context of time. FIG. 8C shows a column towards the right labeled “Dx/context.” If the user clicks on one of these links, the ECCD system will display more contextual information for the data displayed, for example, as is shown in FIG. 8D.

In this example, the user has clicked on the “Dx” link 815 associated with the 65% stenosis in the LAD artery. FIG. 8D shows an example screen displayed by the ECCD system, which may be generated in response to the user interaction with link 815. In this example, when user clicks or activates link 815, a request is transmitted to the ECCD server to request detailed information concerning the selected data item LAD. The request may include a data item identifier identifying the selected data item LAD. In response, the ECCD server retrieves any information related to the selected data item LAD and may perform an analysis on the related information. In one embodiment, the ECCD server determines a likelihood of an abnormal medical condition or disease may occur of the associated patient based on the analysis. The determination may be performed in view of medical data of other patients. In addition, a graph may be generated illustrating the patient's medical data in view of other patients' medical data.

In this example, as shown stage 714 in FIG. 8D, graph 820 shows data relating to this patient in the context of data from other patients with some levels of stenosis. In this example, this patient's data point is indicated with one type of indicator 825 (e.g., a circle) and shows that based on the data from other patients indicated by other types of indicators such as indicator 830 (e.g., solid dots), this patient may have a 33% increased risk of heart disease, based on the 65% stenosis in the LAD. Although this graph shows % stenosis on the left axis, other factors may be incorporated including location of stenosis, age of stenosis, growth rate of stenosis, etc. Other data may also be considered, including symptoms, age of patient, weight, medical history etc. As can be seen, almost any data can be analyzed in conjunction with any other data to determine possible diagnoses and/or treatments. The ECCD system may also search the various databases for similar cases and display for the user cases similar to the one he/she is viewing. The user and/or the ECCD system can analyze similarities and differences. If the ECCD system analyzes the differences and similarities, these may be identified or highlighted in some way in the viewer.

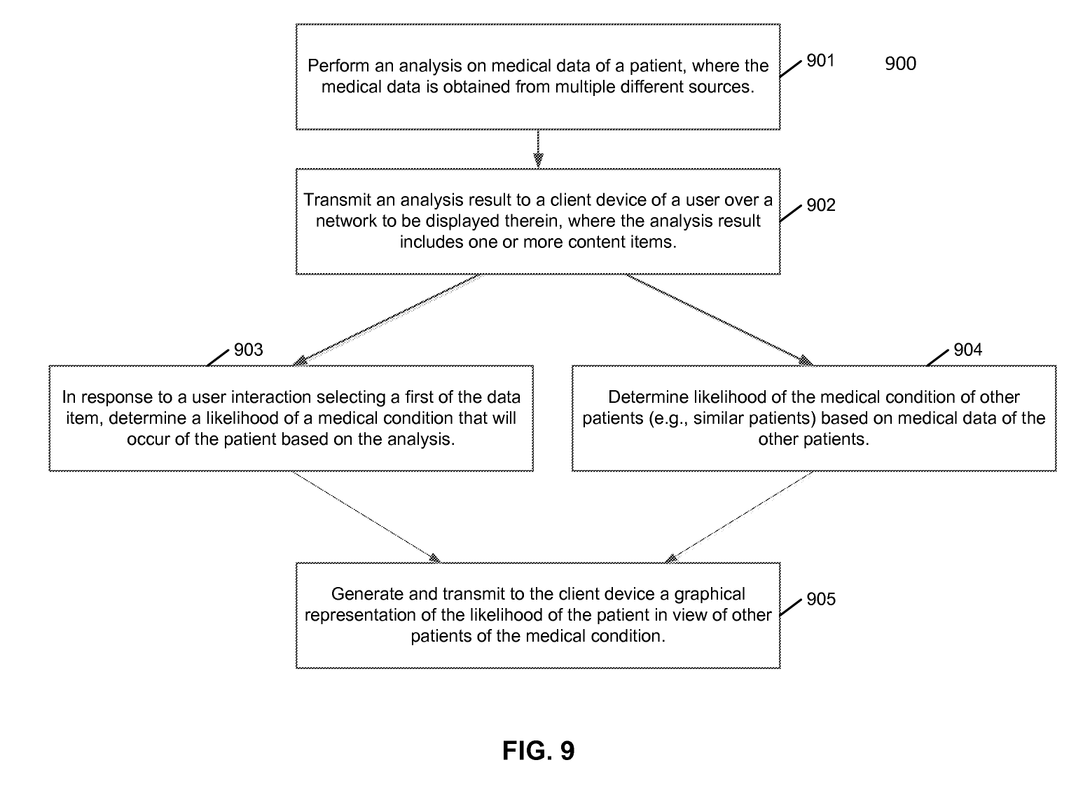

FIG. 9 is a flow diagram illustrating a process for processing medical data using evolving contextual clinical data technology according to another embodiment of the invention. Process 900 may be performed by processing logic, which may include software, hardware, or a combination thereof. For example, process 900 may be performed by medical information server 101. Referring to FIG. 9, at block 901, processing logic performs an analysis on medical data of a patient, where the medical data is obtained from different data sources in response to a request. At block 902, the analysis result is transmitted to a client device of a user over a network to be displayed therein. The analysis result includes one or more content or data items. At block 903, in response to a user interaction selecting a first of the data item, processing logic determines a likelihood or probability of an abnormal medical condition or disease that may occur of the patient based on the analysis. At block 904, processing logic determines the likelihood or probability of the abnormal medical condition or disease of other patients based on the medical data of the other patients. At block 905, processing logic generates and transmits to the client device of the user a graphical representation of the likelihood of the patient in view of other patients of the likelihood of the abnormal medical condition or disease.

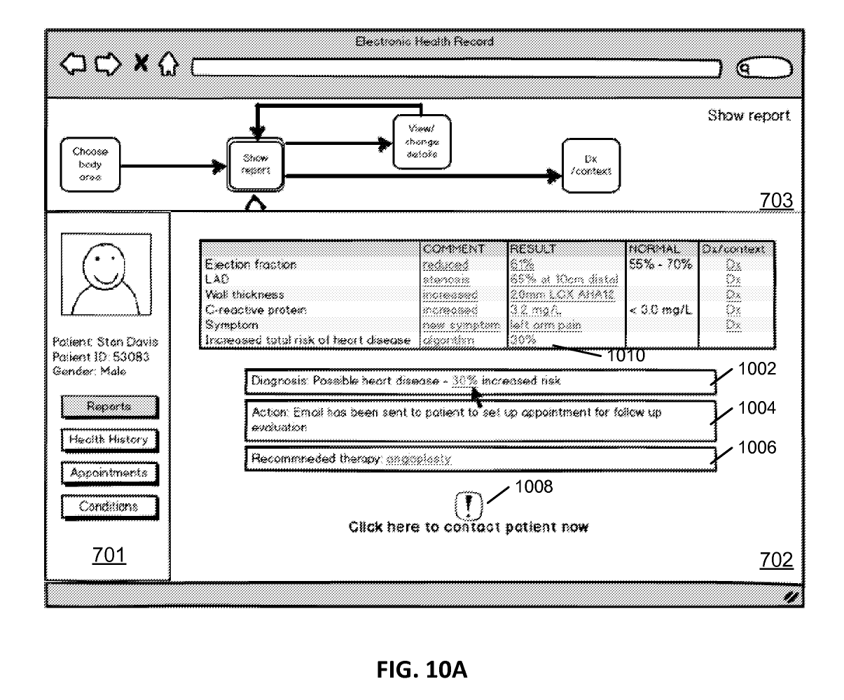

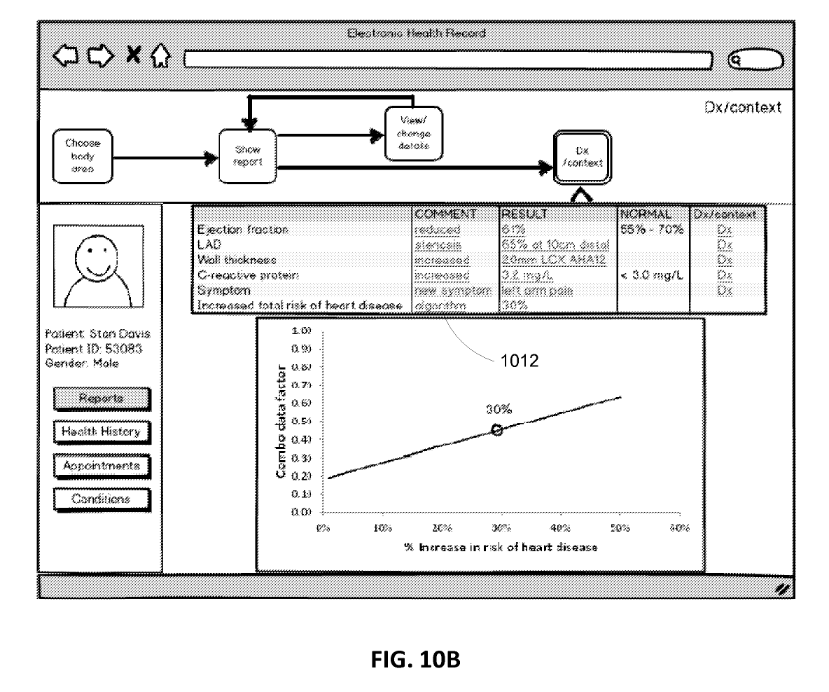

FIGS. 10A and 10B are screenshots representing graphical user interfaces for providing medical information according to some embodiments of the invention. For example, FIGS. 10A and 10B may be generated and displayed in response to a user interaction with FIGS. 7A-7B. Referring to FIG. 10A, FIG. 10A shows another possible report screen which is displayed by the ECCD system. This screen shows data similar to FIGS. 8A and 8C, but in addition, data relating to possible diagnoses, treatment, and next steps are displayed as a recommendation of course of action. Box 1002 shows a diagnosis to the user that based on underlying data and analysis, the ECCD system has determined that this patient has a 30% increased risk of heart disease. The diagnosis may be automatically performed based on the analysis of the patient's data in view of some benchmarks and/or patient's medical history and/or other patients' data, which may be configured as a set of rules.

Box 1004 displays a possible next step, in this case the ECCD system has already emailed the patient to set up a follow-up appointment. The ECCD system can automatically perform an action, in this example, sending a notification to patient, according to a set of rules based on the analysis or diagnosis of the medical data of the patient. Other next steps might include obtaining a lab test, consulting with a specialists etc. Box 1006 displays recommended therapy, in this case angioplasty. The word “angioplasty” is linked here and clicking on it would bring the user to another screen showing details of data and analyses underlying this recommendation.

In this embodiment, the ECCD system performs an analysis on medical data of a patient, including determining a likelihood of an abnormal medical condition or disease based on the analysis as shown in box 1002. In addition, the ECCD system may automatically perform an action according to a set of rules, which can be previously configured. For example, a user may configure that if certain medical data exceeds or drops below a threshold, a notification may be automatically sent to a preconfigured recipient, in this example, the patient. Furthermore, a recommendation is also determined by the ECCD system automatically based on the analysis of the medical data of the patient. All these information can be compiled and integrated into one or more views of medical information and transmitted from the ECCD system to the client device to be displayed therein as shown in FIG. 10A.

FIG. 10A also shows button 1008 which allows the user to contact the patient immediately. This may be useful if the diagnosis and/or treatment is time sensitive in nature. In this example, the diagnosis of a 30% increased risk in heart disease has been determined by the ECCD system by incorporating more than one type of data. For example, the 30% may have been determined by incorporating some or all the data displayed here: ejection fraction, stenosis percent and location, wall thickness, relevant lab test results, symptoms etc. Very complex algorithms may be used to determine the diagnosis. If a user clicks on the diagnosis as shown here, the ECCD system will display more detail relating to the data and/or algorithm behind the diagnosis. FIG. 10B shows some detail regarding how the 30% increase in risk of heart disease diagnosis may be determined by the ECCD system, for example, in response to user interaction with link 1010 of FIG. 10A. In this example, by way of a graph. Clicking on algorithm link 1012 would show the user more details behind the algorithm.

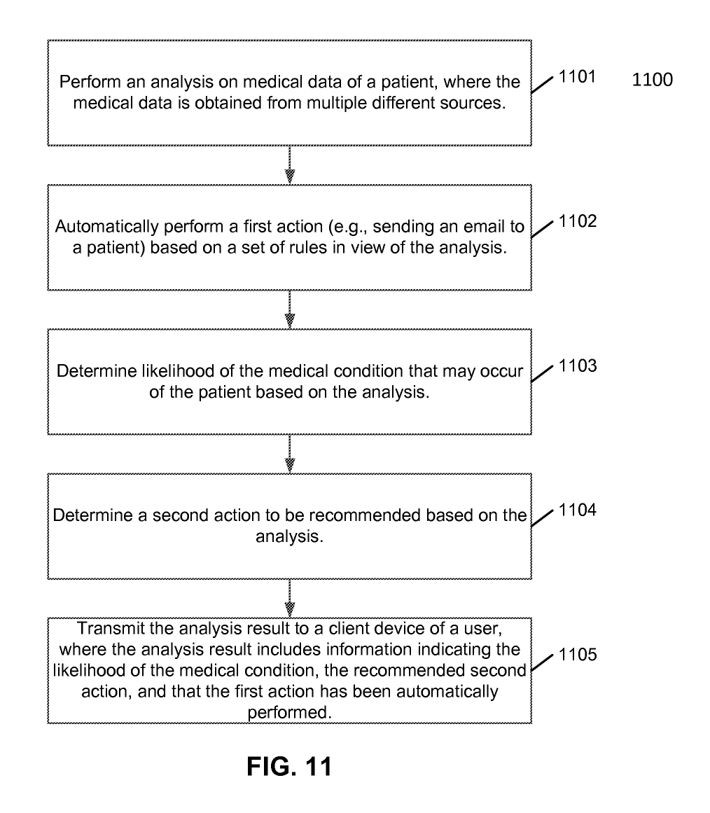

FIG. 11 is a flow diagram illustrating a process for processing medical data using evolving contextual clinical data technology according to another embodiment of the invention. Process 1100 may be performed by processing logic, which may include software, hardware, or a combination thereof. For example, process 1100 may be performed by medical information server 101. Referring to FIG. 11, at block 1101, processing logic performs an analysis on medical data of a patient, where the medical data may be obtained from different data sources provided by different information providers or servers. The analysis may be performed based on patient's other data such as lab reports, medical history, etc. and medical data of other patients. Based on the analysis, at block 1102, processing logic automatically performs a predetermined action (e.g., sending a notification to a preconfigured recipient) based on a set of rules. For example, for a particular medical data item or data type, a user can configure a set of rules that if that particular data item exceeds or drops below a predetermined threshold or a well-known benchmark, a predetermined action should be performed. At block 1103, processing logic determines a likelihood that an abnormal medical condition or disease may occur for the patient based on the analysis. Again such a determination may be based on the patient's other medical data and/or medical history, as well as other patient's similar medical data. At block 1104, processing logic determines a second action to be performed as a recommendation based on the analysis. At block 1105, the analysis result is transmitted to the client device to be displayed therein. The analysis result includes information indicating that the first action has already performed, the likelihood of an abnormal medical condition or disease, and the recommendation.

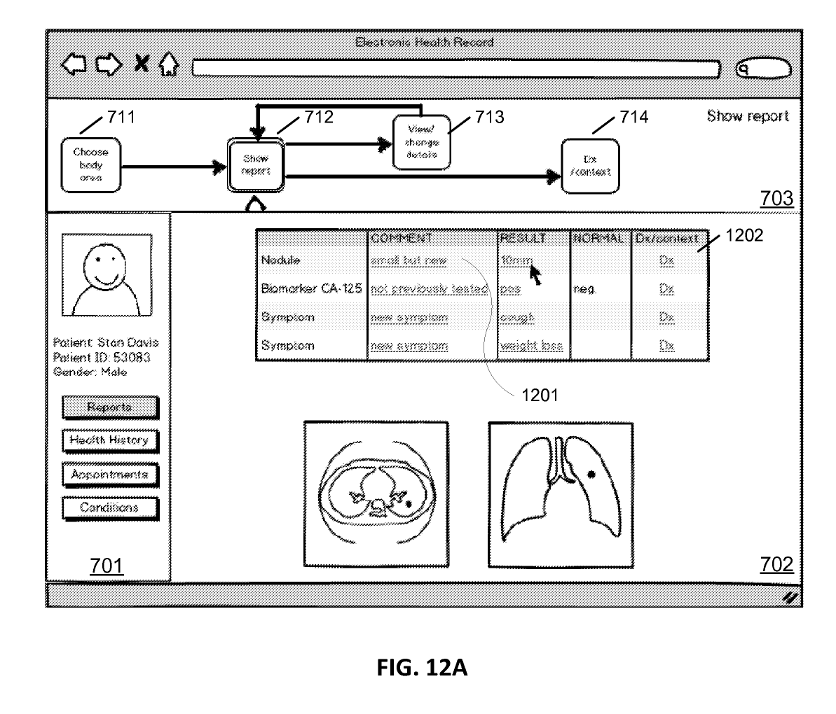

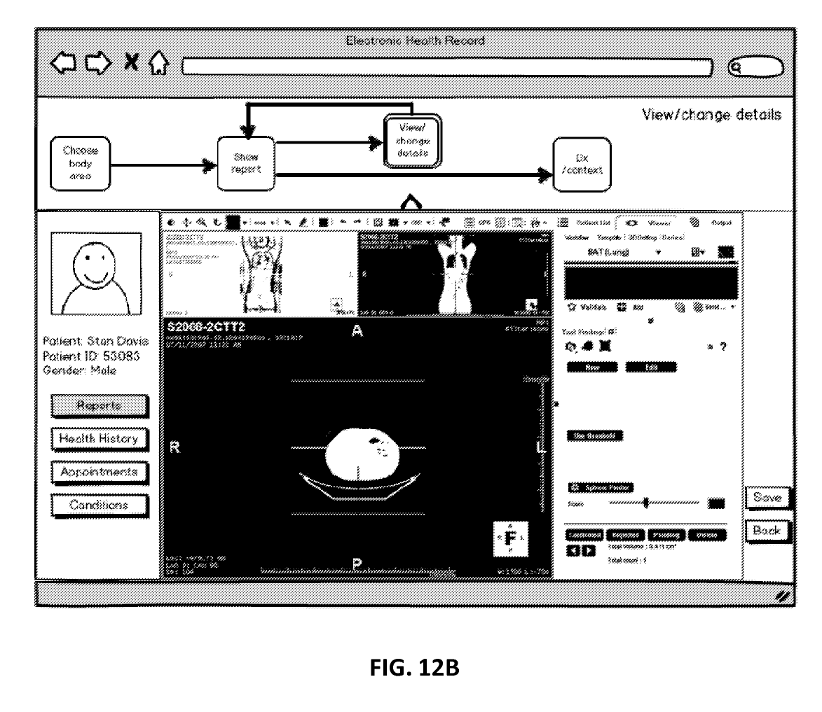

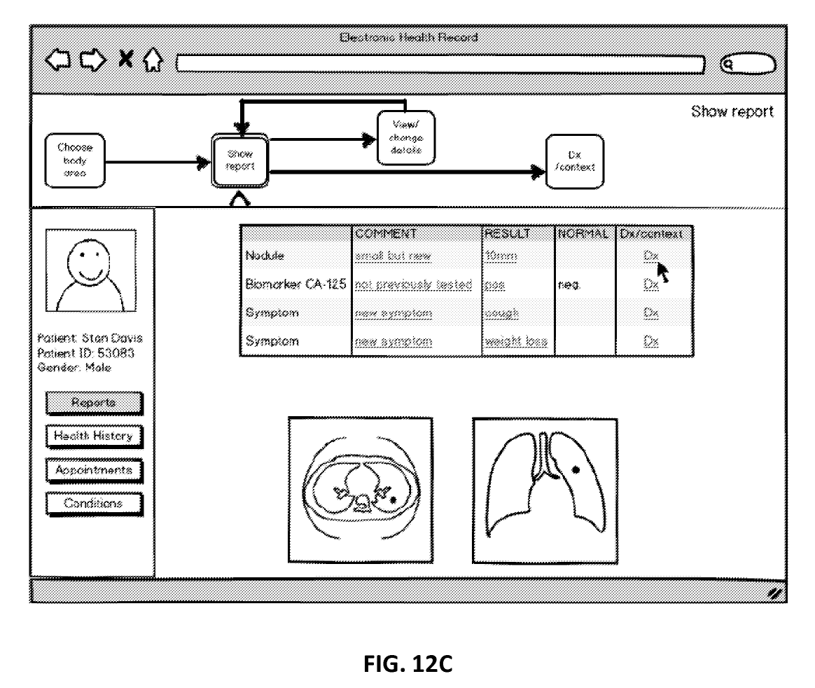

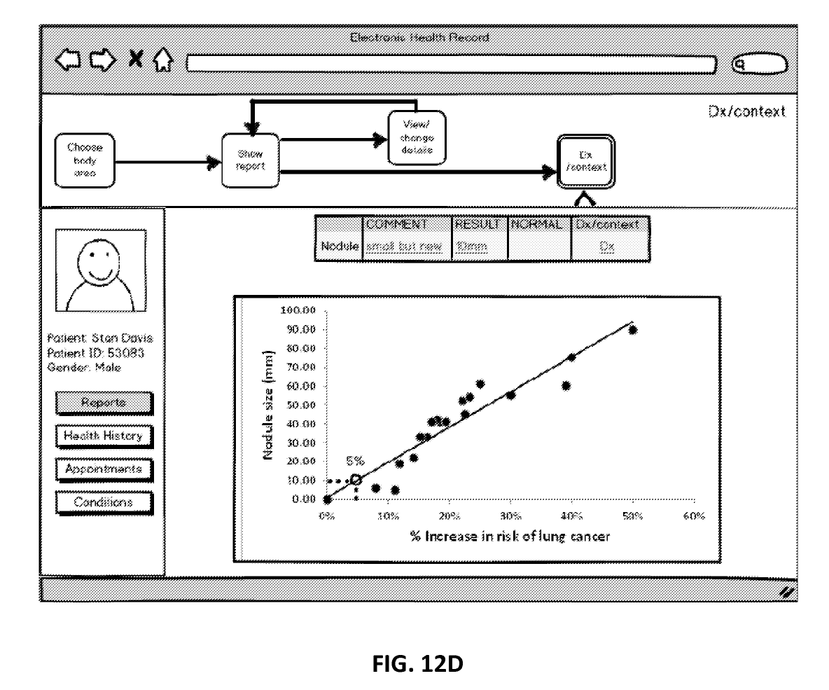

FIGS. 12A-12D are screenshots representing graphical user interfaces for providing medical information according to some other embodiments of the invention. For example, FIGS. 12A-12D may be generated and displayed in response to a user interaction with FIGS. 7A-7B. Referring to FIG. 12A, the GUI page may be generated and displayed in response to a user interaction that selects a lung from FIG. 7A. In this example, when a user selects the lung, for example, from the GUI page as shown in FIG. 7A, a request is transmitted by a client application (e.g., Web browser) from the client device to the ECCD server over a network. The request may include a body part ID identifying a lung and a patient ID identifying a patient in question. The request may further include a user ID identifying the user who initiates the request. Such information allows the ECCD server to determine (e.g., based on access control list or ACL of the user) whether the user is entitled to access the patient information of that particular patient and if so, what kinds of medical data of the patient the user is entitled to access.

In response to a user selection of a lung associated with a patient, the ECCD server retrieves any medical data that is related to the lung of a patient identified by a body part ID identifying the lung of the patient identified by a patient ID. In this example, the data is displayed within a frame, tab, or area of a web browser, although the data may be displayed in a separate window or pop-up window. The data displayed may be data which is stored in various different data sources, such as the RIS, PACS, LIS, etc. the ECCD system may also have performed further analysis on the data. For example, in the table a lung nodule diameter is displayed as 10 mm. This number may be determined by the ECCD system by analyzing image series data, for example, CT scan image series data. Also, comparing results to normal ranges may be part of the analysis and display.

The data displayed by the ECCD system in this sample table include data from images, image analysis, lab reports, and symptoms. These data generally reside in very different server systems and databases. The ECCD system is able to associate the relevant data with this patient, and this body part, the lung, perform any relevant analyses, and present the relevant data to the user in the viewer in context. The viewer may be stand-alone, or may be integrated with another software system such as an EHR software system or clinical trials software system.

In this example, the ECCD system analyzes CT scan data and determines that patient X has a lung nodule which is new and 10 mm in diameter. The ECCD system may know the nodule is new by analyzing CT scan data over time. The system may also access lab data relating to cancer, as well as symptom data and physician report/dictation/notes data which relates to the lung and/or to cancer. For example, the ECCD system may determine that the CA-125 biomarker, relating to cancer, is positive. The ECCD system may also have detected the symptom “cough” in recent physician notes, but not in previous notes/symptoms/reports, and display that “cough” is a new symptom. The ECCD system may also have analyzed patient history/demographic data over time and determined that this patient has lost weight recently.

If the user clicks on the linked nodule diameter data 1201, 10 mm, the ECCD system may display a page similar to FIG. 12B. Similar to FIG. 8B in the heart example, FIG. 12B shows advanced image processing results and tools for the lung. Here the user can check how the 10 mm result was determined, look at earlier CT scans to see how they compare etc. The user can also make changes to the analyses if he/she has the appropriate access level to do so. FIG. 12C is identical to FIG. 12A except that it shows where a user may click to review the Dx/context of the lung nodule which is shown in FIG. 12D.

FIG. 12D is similar to FIG. 8D in that the ECCD system displays context for this patient's lung nodule data. Here Patient X's data point is displayed in the context of several other patients who have had lung nodules. Based on this data pool, the ECCD system has determined that a lung nodule of 10 mm presents a 10% increase risk of lung cancer. Although this analysis only takes into consideration the nodule diameter, other factors could also be considered, including nodule density, nodule shape, nodule location, nodule volume, nodule growth rate, symptoms, lab tests etc. The ECCD system may also determine the patient's likelihood to live for certain time frames, such as 5 years, 10 years etc., based on the data.

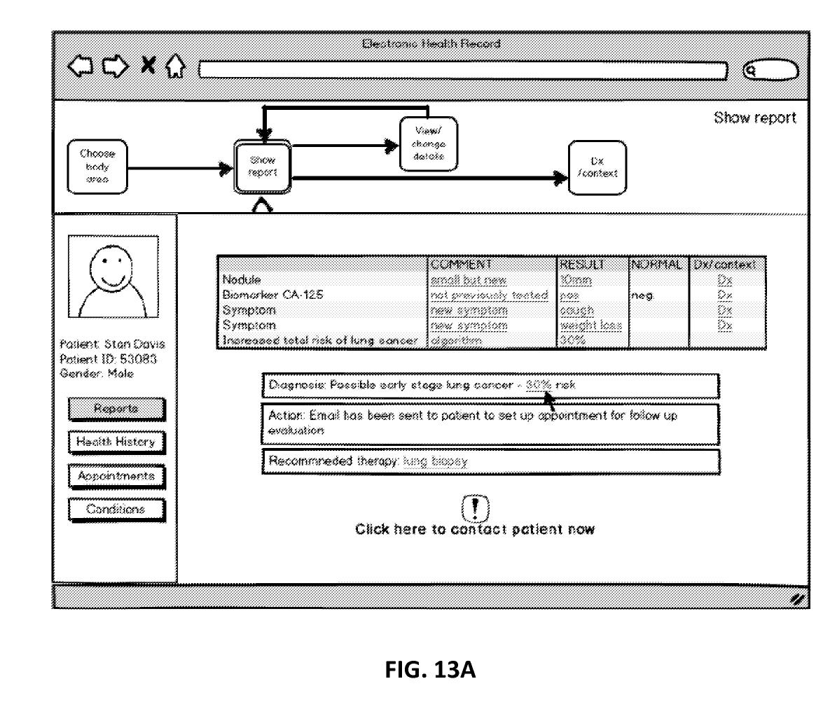

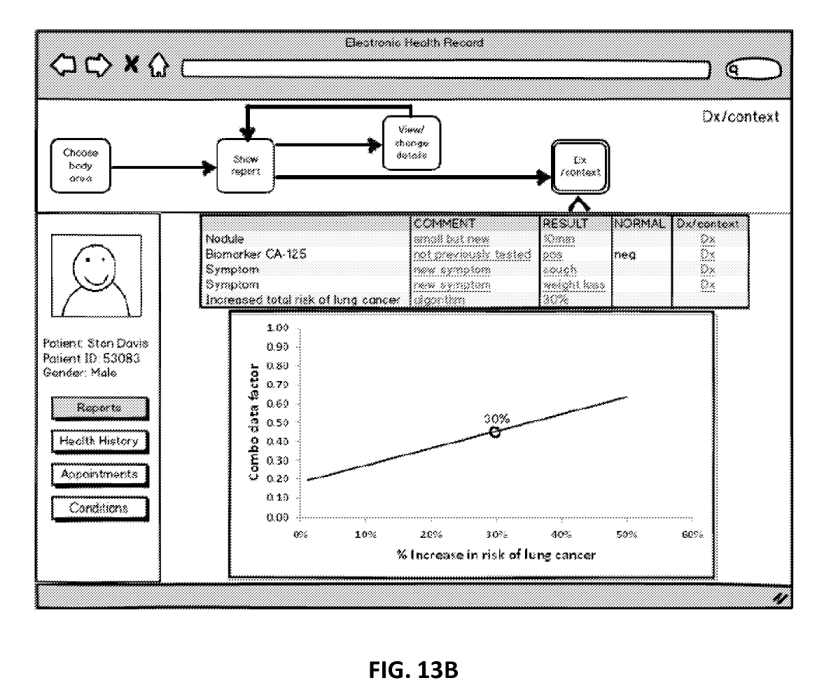

FIGS. 13A-13B are screenshots representing graphical user interfaces for providing medical information according to some other embodiments of the invention. For example, FIGS. 13A-13B may be generated and displayed in response to a user interaction with FIGS. 7A-7B. Referring to FIG. 13A, FIG. 13A shows the results of the ECCD systems analysis including diagnosis, next steps and recommended therapy. FIG. 13B shows data relating to a diagnosis which is determined by an algorithm incorporating more than one data point. For example, biomarker test results, symptoms, nodule growth rate and size etc.

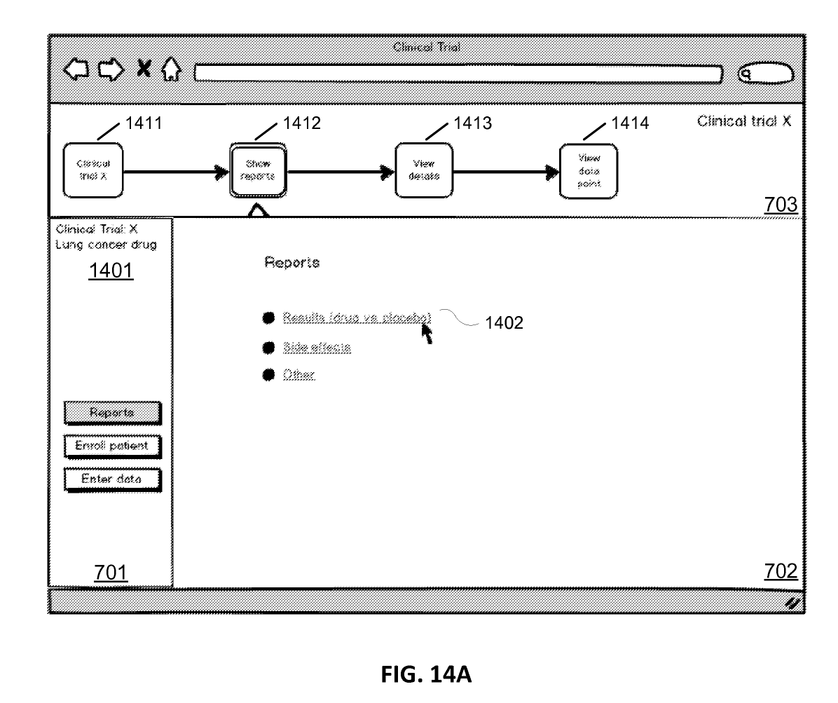

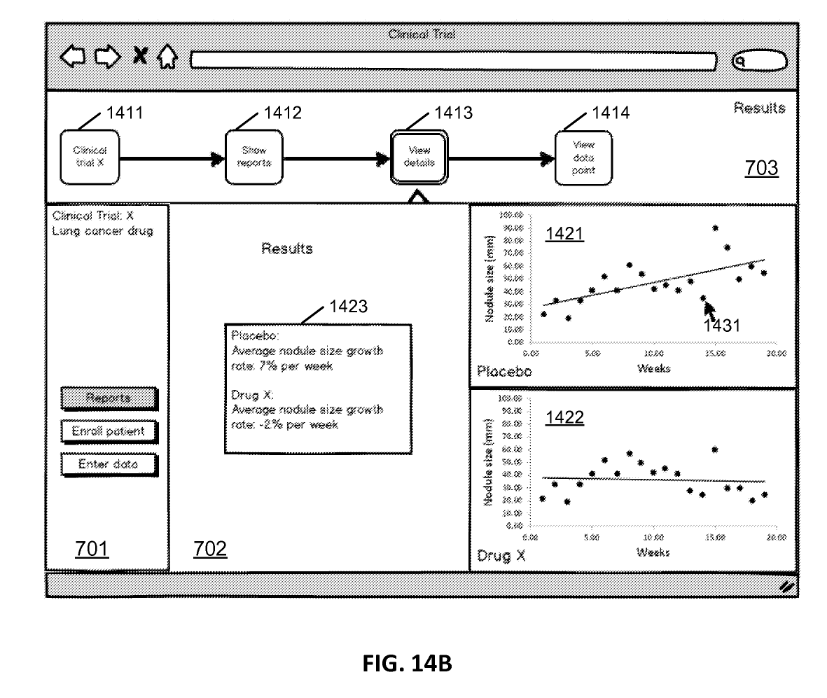

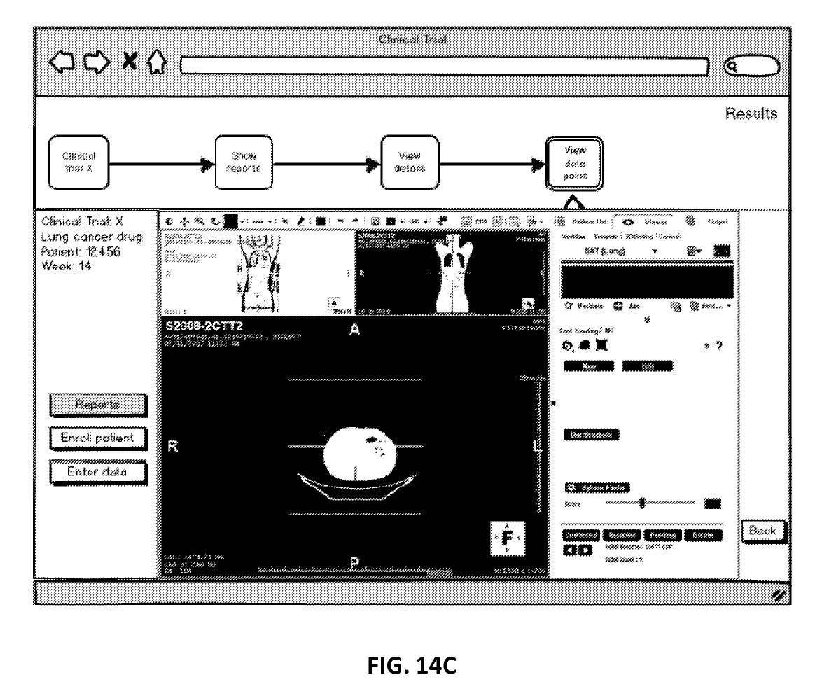

FIGS. 14A-14C are screenshots representing graphical user interfaces for providing medical information according to some other embodiments of the invention. Referring to FIG. 14A, FIG. 14A shows an example of the ECCD system in conjunction with a different type of medical software. In this example, the ECCD system is being integrated with a clinical trial software system rather than an EHR software system, which includes processing stages 1411-1414 displayed in display area 703.

Clinical trial label 1401 shows which clinical trial the user is viewing, in this example “Clinical Trial X”. Workflow or step timeline in display area 703 shows where the user is in the process, in this example, in the “show reports” step 1412. Several reports may be available to the user. In this example, the user clicks on the “results” report, which transitions from stage 1412 to stage 1413 as shown in FIG. 14B. FIG. 14B shows an example screen displaying results. Graphs 1421 and 1422 show graphs of lung nodule size over time with both the placebo and the test drug treatment protocols, both of which the ECCD system displays based on analysis of data, including imaging data. Box 1423 is a higher level display of the current results of the drug vs. placebo results. The ECCD system has displayed this summary based on the analysis of the data in the 2 graphs. The ECCD system may display other types of data including side effects, effect of other medications on the results, breakdown of results by age, sex, etc., dosages, and/or any other relevant data. The viewing areas may be in frames and/or tabs within a web browser, or separate windows or on separate monitors. The viewing areas may overlap, or be integrated with each other. The viewing areas may be within one another data and may also be linked to more detailed information relating to the data. For example, data point 1431 may be clickable to view the detailed information and analysis behind the data point 1431, as shown in FIG. 14C. FIG. 14C shows a possible screen displayed by the ECCD system if a user clicks on data point 1431 in FIG. 14B. FIG. 14C shows detailed image scan data and analysis. The data may also be changed here, but in the example of a clinical trial, normally access level to change data would be much more limited than in the example of data displayed in the context of an EHR.

The ECCD system may also learn. This learning may take place either across users and/or data, or within certain subsets or data or within one user or user type. For example, the ECCD system may collect user data that shows that users generally do not click on lab data when viewing a colposcopy case, and place lab data on a lower display priority when future users view colonoscopy cases. In another example, the ECCD system may collect data that a particular user views EKG data for a longer period of time than image data, and the ECCD system may place EKG data at a higher display priority for this user.

Referring back to FIG. 2, in one embodiment, medical imaging processing system 210 includes an image processing engine which provides medical image processing services to clients over a network. The image processing engine may be implemented using dedicated graphics or image processing hardware, such as graphics processing units (GPUs). Medical imaging processing system 210 also includes or is associated with an image store (not shown) to store medical data such as digital imaging and communications in medicine (DICOM) compatible data or other image data, including JPEG, TIFF, video, EKG, laboratory images, reports, text, PDF, sound, and other files. The image store may also incorporate encryption capabilities, where the medical data can be stored and transmitted in an encrypted form. The image store may include multiple databases, and may be implemented with relational database management systems (RDBMS), e.g., Oracle™ database or Microsoft® SQL Server, etc.

In one embodiment, the medical information server 101 includes an access control system (not shown) to control access, by the client device 102, of resources (e.g., image processing tools) and/or medical data stored in image store. Clients 102 may or may not access certain portions or types of resources and/or medical data stored in image store depending upon its access privilege. The access privileges may be determined or configured based on a set of role-based rules or policies. For example, client 102 may be configured with certain roles that only permit access to some of the tools provided by the medical information server 101. In other instances, the client may be configured with certain roles that limit its access to some patient information. For example, certain users (e.g., doctors, medical students) of client 102 may have different access privileges to access different medical information stored in image store 108 or different imaging rendering resources provided by medical information server 101.

Client devices 102 may be a client which may include integrated medical software. In one embodiment, the integrated software integrates image(s) and/or image processing functionality with medical record software (MRS) and/or clinical trial software (CTS), which herein are collectively referred to as medical record and/or clinical software (MRCS). Medical record software (MRS) is patient-centric software that focuses on medical records of the individual patients. Patient-centric means here that the software's primary purpose is to record and view data relating to the individual patient. This type of software may be referred to as electronic medical record (EMR) software, electronic health record (EHR) software, personal health record (PHR) software and other names. Information maintained by the MRS typically includes: patient ID, demographic, info—age, weight, height, Blood Pressure (BP), etc., lab orders and results, test orders and results, medical history, appointment history, appointments scheduled, exam history, prescriptions/medications, symptoms/diagnoses, and insurance/reimbursement info.

Clinical trial software (CTS) includes software for both retrospective and prospective clinical studies. This type of software may be referred to as a clinical trial management system. CTS may also include software for research. CTS is trial-centric which means the primary purpose of the software is to collect and view aggregate data for multiple patients or participants. Although data is collected at the individual patient/participant level, this data is usually viewed “blindly”. This means that the viewer and/or analyzer of the data generally do not know the identity of the individual patients/participants. However, data can be viewed at the individual patient/participant level where necessary. This is particularly important where images are involved. CTS typically includes: patient ID, concomitant medications, adverse events, randomization info, data collection, informed consent, aggregated data, and status of study.

In one embodiment, client application 207 running as integrated medical software executed within the client 102A displays medical information of a patient, including, e.g., the medical treatment history of a patient, which may be part of a medical record and/or trial record of the patient. Such records may be downloaded from medical information server 101 in response to a user request and/or recommended by ECCD engine 103. In the case where the integrated medical software integrates MRS, the patient's full identity it typically displayed as part of the medical information. On the other hand, in the case of an integrated CTS, the patient is typically anonymous as discussed above, and the identity of the patient is typically not revealed as part of the displayed medical information.

In one embodiment, image(s) and/or image processing functions may be integrated with the MRCS. Integration can take the form of the image(s) and/or image processing tools showing up in the same window as the MRCS. Integration can also take the form of a window containing the image(s) and/or image processing tools opening up in a separate window from the MRCS window. It should be noted, however, that in either form of integration, the medical information of the patient and image(s) are displayed within the integrated medical software, without requiring the user of the integrated software to separately obtain the images via another software program.

In one embodiment, medical image processing system 210 includes an advanced image processing system and an automatic image processing system (e.g., image processing wizard). When the advanced image processing system is utilized, a set of graphical representation representing a set of image processing tools may be presented in an advanced image processing graphical user interface to allow a user to specify one or more of the image processing tools to process a particular one of images. When the automatic image processing system is utilized, the underlying processing logic of the automatic image processing system is configured to automatically determine and select one or more image processing tools to process the image, for example, without user intervention or user knowledge of which of the image processing tools to be utilized. The graphical representations (e.g., icons) for image processing tools that are provided by the remote imaging processing server are displayed to the user of the integrated medical software executed on the client. In such an embodiment, the available image processing tools are displayed in the integrated medical software as a set of icons or some other graphical representations, which when activated by a user, allow an image to be manipulated by remote imaging processing system 210. In one embodiment the image processing software is integrated with the MRCS program and also opens up “in context.” “In context” means that the image processing software opens up to show the appropriate image(s) and/or tools for the current user and/or patient and/or affliction. The availability of imaging tools to a particular user depends on the access privileges of that particular user (e.g., doctors vs. medical students). Alternatively, the availability of imaging tools may be determined based on a particular body part of a patient, which may be identified by certain tags such as DICOM tags.

For example, one doctor may prefer that the cardiovascular images for his patients open up in a 3D view, with vessel centerline tools available, yet the abdominal images for his patients open up in a coronal view with the flythrough, or virtual colonoscopy, tools available. He may prefer to have the other views and tools hidden from view. In another example, another doctor may prefer that the images for her patients open up showing the most recent views and tools that she used for that patient. In another example, the default view for cardiovascular cases may be set to show a particular view and tools, but the user may be able to change the default so that his/her preferences override the default views and tools.

In all of the above examples, ideally only the images that relate to the patient being evaluated at that time are able to be viewed. In addition, the user/physician does not need to search to find the images relating to the patient, the images are automatically associated with the correct patient, for example, based on the corresponding patient ID. To do this, the identity of the patient needs to be associated with the patient's images. This can be done by using tags, such as a common identifier, such as an ID number, metadata associated with one or more of the images, mining patient data, body part analysis, or other ways. Also, the appropriate tools need to be shown and inappropriate tools hidden. The tags are discussed in more details below.

For example, an image or image series can be analyzed to determine whether it is a head, abdomen, or other body part, based on the anatomy. A skull has a characteristic shape, as do other parts of the anatomy. A catalog of reference images may be used to help identify specific body parts. Based on this analysis, the appropriate views and/or tools can be made visible to the user, and inappropriate views and/or tools can be hidden. For example, if the image series is of a head/skull, the image series may be shown in a certain view, such as an axial view, and tools associated with the brain visible. In addition, if certain key words, such as “tumor” or “stroke”, are found in the MRCS record, specific tools may be shown, such as tools that detect a tumor or evaluate brain perfusion. It is also possible that a patient ID can be determined from the anatomy in an image based on shape, disease, tags etc. For example, an image of a dental area can be matched with dental records to identify a patient from medical images. Or, an identifying tag can be included in the medical image—such as a tag with the patient ID number placed on or near the table of a CT scanner, or on the patient himself. In another embodiment, the user of the software is able to customize how the image processing software is presented in context. For example, Doctor Y, a cardiologist, may prefer to have the images open up in a 3D model view, and have cardiology tool A and cardiology tool B visible to him. In this example, other views may be hidden (for example, the axial, sagittal, and coronal views) and other tools are hidden (for example, tools relating to the colon or the brain).

According to one embodiment, the advance image processing system allows users of different types to access the imaging tools represented by tool icons for processing images, which utilize processing resources (e.g., image processing engine) over the network. The automatic image processing system allows users of different types to access the functionality of imaging tools without having to deal with the tools directly. The automatic image processing system may be layered on top of, or integrated with, an existing or new advanced medical image processing software system (e.g., advanced image processing system) to simplify or automate the use of the medical image processing resources (e.g., image processing engine), which may be implemented in software, hardware, or a combination of both.

According to one embodiment, both the advanced image processing system and the automatic image process system may access the image processing functions (e.g., libraries, routines, tools, etc.) of the underlying image processing engine via a set of application programming interfaces (APIs) or communication protocols. When the advanced image processing system is utilized, according to one embodiment, an advanced graphical user interface may be presented to allow the user to specify detailed image processing parameters for processing a specific image selected by the user. The underlying processing logic of the advanced image processing system processes the user inputs received from the advanced graphical user interface and formulates one or more image processing commands with a set of image processing parameters that are generated based on the user inputs. The processing logic of the advanced image processing system then sends the commands, for example, via the APIs, to the backend image processing engine to process the image.

When the automatic image processing system is utilized, according to one embodiment, a simplified graphical user interface (e.g., wizard) is presented at a client device of the user to walk the user through a series of simple steps or interactive questions without requiring the user to specify the detailed operational image processing parameters. The underlying processing logic is configured to automatically determine the detailed image processing parameters based on the user interaction with the simplified graphical user interface. A set of image processing commands is generated and sent to the backend image processing engine for processing the image. Alternatively, the underlying processing logic of the automatic image processing system determines the parameters and passes the parameters to the advanced image processing system, just as the advanced image processing system would have received from a user via its corresponding graphical user interface. The advanced image processing system in turn communicates with the underlying image processing engine on behalf of the automatic image processing system.

The automatic image processing system may be implemented in a form of an image processing wizard. The wizard guides a user through the advanced image processing process. The wizard automates as many steps as possible, for example, using preferences, assumptions, and a set of rules, to process image data, such that the user does not have to know the details of how to operate the advanced image processing tools. The wizard also gives the user an opportunity to confirm or change the results that were created automatically or otherwise. The wizard may consist of the presentation of intuitive user interfaces as well as easy to answer questions which help guide the user through the image processing process.

According to one embodiment, the automatic image processing system provides a user friendly interactive graphical user interface. The automatic image processing system allows a user to access the underlying processing resources based on a set of user understandable processing stages to perform certain major or common or popular image processing operations on an image, without having to fully understand specific steps and/or image processing parameters or tools for processing the image. The automatic image processing system, through a user friendly graphical user interface (GUI), may interact with the user through a series of questions and receive user inputs as a part of answers from the user to determine the user's intent. Based on the user interaction with the automatic image processing system, one or more image processing operations may be determined and recommended to the user via the automatic image processing system. The user can select one or more of the recommended image processing operations for processing the image, or alternatively, image processing operations may be performed automatically by the automatic image processing system. Based on a user selection of one or more of the image processing indicators, one or more image processing parameters associated with the selected image processing operations are automatically determined without user intervention and without having the user providing the same parameters.

Based on the image processing parameters received by the automatic image processing system, according to one embodiment, one or more image processing commands are generated and transmitted from the automatic image processing system to the image processing engine for image processing. In response to the image processing commands, the image processing engine processes the image based on the image processing parameters and generates a new or updated image. The new image may represent a different view of the same medical data associated with the original image. The new image is then transmitted from the image processing engine back to the automatic image processing system, which in turn transmits the new image to the client device to be presented to the user. The automatic image processing system also causes the client to prompt the user whether the user is satisfied with the new image. If the user is unsatisfied with the new image, the automatic image processing system may interact with the user for more user inputs concerning the new image and further adjust the image processing parameters and the image processing operations may be iteratively performed. As a result, a user does not have to fully understanding how to utilize the advanced image processing system, although the advanced image processing system may also be available for advanced users.

According to one embodiment, in response to image data received from a medical data center or from image capturing devices (not shown) or from another image source, such as a CD or computer desktop, according to one embodiment, image preprocessing system 210 may be configured to automatically perform certain preprocesses of the image data and store the preprocessed image data in a medical data store (not shown). For example, upon receipt of an image data from PACS or directly from medical image capturing devices, image preprocessing system 204 may automatically perform certain operations, such as bone removal, centerline extraction, sphere finding, registration, parametric map calculation, reformatting, time-density analysis, segmentation of structures, and auto-3D operations, and other operations, some of which are listed later herein.

Image preprocessing system 210 further a workflow management system. The workflow management system may be a separate server or integrated with image processing system 210. The workflow management system performs multiple functions according to some embodiments of the invention. For example, the workflow management system performs a data server function in acquiring and storing medical image data received from the medical image capturing devices. It may also act as a graphic engine or invoke image processing system 210 in processing the medical image data to generate 2D or 3D medical image views. In one embodiment, the workflow management system invokes image processing system 210 having a graphics engine to perform 2D and 3D image generating. When a client requests for certain medical image views, the workflow management system retrieves medical image data stored in a medical data store, and renders 2D or 3D medical image views from the medical image data. The end results for medical image views are sent to the client.

In one embodiment, the workflow management system manages the creation, update and deletion of workflow templates. It also performs workflow scene creation when receiving user requests to apply a workflow template to medical image data. A workflow is defined to capture the repetitive pattern of activities in the process of generating medical image views for diagnosis. A workflow arranges these activities into a process flow according to the order of performing each activity. Each of the activities in the workflow has a clear definition of its functions, the resource required in performing the activity, and the inputs received and outputs generated by the activity. Each activity in a workflow is referred to as a workflow stage, or a workflow element. With requirements and responsibilities clearly defined, a workflow stage of a workflow is designed to perform one specific task in the process of accomplishing the goal defined in the workflow. For many medical image studies, the patterns of activities to produce medical image views for diagnosis are usually repetitive and clearly defined. Therefore, it is advantageous to utilize workflows to model and document real life medical image processing practices, ensuring the image processing being properly performed under the defined procedural rules of the workflow. The results of the workflow stages can be saved for later review or use.

In one embodiment, a workflow for a specific medical image study is modeled by a workflow template. A workflow template is a template with a predefined set of workflow stages forming a logical workflow. The order of processing an activity is modeled by the order established among the predefined set of workflow stages. In one embodiment, workflow stages in a workflow template are ordered sequentially, with lower order stages being performed before the higher order stages. In another embodiment, dependency relationships are maintained among the workflow stages. Under such arrangement, a workflow stage cannot be performed before the workflow stages it is depending on being performed first. In a further embodiment, advanced workflow management allows one workflow stage depending on multiple workflow stages, or multiple workflow stages depending on one workflow stage, etc.

The image processing operations receive medical image data collected by the medical imaging devices as inputs, process the medical image data, and generate metadata as outputs. Metadata, also known as metadata elements, broadly refers to parameters and/or instructions for describing, processing, and/or managing the medical image data. For instance, metadata generated by the image processing operations of a workflow stage includes image processing parameters that can be applied to medical image data to generate medical image views for diagnostic purpose. Further, various automatic and manual manipulations of the medical image views can also be captured as metadata. Thus, metadata allows the returning of the system to the state it was in when the metadata was saved.

After a user validates the results generated from processing a workflow stage predefined in the workflow template, the workflow management system creates a new scene and stores the new scene to the workflow scene. The workflow management system also allows the updating and saving of scenes during user adjustments of the medical image views generated from the scenes. Further detailed information concerning the workflow management system can be found in co-pending U.S. patent application Ser. No. 12/196,099, entitled “Workflow Template Management for Medical Image Data Processing,” filed Aug. 21, 2008, now U.S. Pat. No. 8,370,293, which is incorporated by reference herein in its entirety.

As described above, a variety of image processing tools can be accessed by a user using the image processing system. The following are examples of medical image processing tools that may be included as part of the image processing system described above. These examples are provided for illustrative purposes and not intended to be a limitation of the present invention.

Vessel Analysis tools may include a comprehensive vascular analysis package for CT and MR angiography capable of a broad range of vascular analysis tasks, from coronary arteries to aortic endograft planning and more general vascular review, including carotid and renal arteries. Auto-centerline extraction, straightened view, diameter and length measurements, CPR and axial renderings, and Vessel Track mode for automated thin-slab MIP may be included.

Calcium scoring tools may include Semi-automated identification of coronary calcium with Agatston, volume and mineral mass algorithms. An integrated reporting package with customization options may be included.

Time-dependent analysis (TDA) tools may include time-resolved planar or volumetric 4D brain perfusion examinations acquired with CT or MR. The TDA tools may support color or mapping of various parameters such as mean enhancement time and enhancement integral, with semi-automated selection of input function and baseline, to speed analysis. TDA tools may support rapid automated processing of dynamic 4D area-detector CT examinations to ensure interpretation within minutes of acquisition.

CT/CTA (Computed tomography angiography) subtraction tools are used in the removal of non-enhancing structures (e.g. bone) from CT angiography examinations, the CT/CTA option includes automatic registration of pre- and post-contrast images, followed by a dense-voxel masking algorithm which removes high-intensity structures (like bone and surgical clips) from the CTA scan without increasing noise, aiding with the isolation of contrast-enhanced vascular structures.

Lobular decomposition tools identify tree-like structures within a volume of interest, e.g. a scan region containing a vascular bed, or an organ such as the liver. The LD tool can then identifies sub-volumes of interest based on proximity to a given branch of the tree or one of its sub-branches. Research applications include the analysis of the lobular structure of organs.

General Enhancement & Noise Treatment with Low Exposure tools may include an advanced volumetric filter architecture applying noise management techniques to improve the effectiveness of 3D, centerline, contouring and segmentation algorithms even when source image quality is not optimum.

The Spherefinder tools perform automated analysis of volumetric examinations to identify the location of structures with a high sphericity index (characteristics exhibited by many nodules and polyps). Spherefinder is often used with Lung or Colon CT scans to identify potential areas of interest.

Segmentation, analysis & tracking tools support analysis and characterization of masses and structures, such as solitary pulmonary nodules or other potential lesions. Tools may identify and segment regions of interest, and then apply measurement criteria, such as RECIST and WHO, leading to tabulated reporting of findings and follow-up comparison. Display and management of candidate markers from optional detection engines may be supported, including Spherefinder.

Time volume analysis tools may provide automated calculation of ejection fraction from a chamber in rhythmic motion, such as a cardiac ventricle. A fast and efficient workflow may be included to enable the user to identify the wall boundaries of interest (e.g. epicardium and endocardium) and, based on these user-confirmed regions of interest, to report ejection fraction, wall volume (mass) and wall thickening from multi-phasic CT data. Tabulated reporting output is included.

Maxillo-facial tools support the analysis and visualization of CT examinations of the Maxillo-facial region, these tools apply the CPR tool to generate “panoramic” projections in various planes and of various thicknesses, and cross-sectional MPR views at set increments along the defined curve plane.

Applicable to endoluminal CT or MR investigations such as colon, lungs, or blood vessels, the Flythrough tools supports side-by-side review, painting of previously-viewed areas, percent coverage tracking, and multiple screen layouts including forward, reverse, fisheye and flat volume rendered views. Tools for contrast subtraction, “Cube View”, and integrated contextual reporting may be supported. Display and management of candidate markers from optional detection engines may be supported, including iNtuition's Spherefinder.

The Volumetric Histogram tools allow a volume of interest to be segmented and analyzed for composition. Research applications include the analysis of low-attenuation regions of the lungs, threshold-based division of tumors into voxel populations, investigation of thrombosed vessels or aneurysms, or other pathology.

Findings workflow tools provide a framework for tracking findings across serial examinations. A database holds measurements and key images, and provides support for structured comparisons and tabulated reporting of findings over time, such as the RECIST 1.1 approach for presenting serial comparisons. The Annotation and Image Markup (AIM) XML schema may be supported, for automated integration with voice-recognition systems or clinical databases, and Word-based reports may be derived from the database.

With these tools, any two CT, PET, MR or SPECT series, or any two-series combination thereof can be overlaid with one assigned a semi-transparent color coding and the other shown in grayscale and volume rendering for anatomical reference. Automatic registration is provided and subtraction to a temporary series or to a saved, third series is possible. Support for PET/MR visualization is included.

Certain MR examinations (for example, Breast MR) involve a series of image acquisitions taken over a period of time, where certain structures become enhanced over time relative to other structures. These tools feature the ability to subtract a pre-enhancement image from all post-enhancement images to emphasize visualization of enhancing structures (for example, vascular structures and other enhancing tissue). Time-dependent region-of-interest tools may be provided to plot time-intensity graphs of a given region.

Parametric mapping tools are an enhancement to the Multi-Phase MR tools, the parametric mapping option pre-calculates overlay maps where each pixel in an image is color-coded depending on the time-dependent behavior of the pixel intensity. As an example, this tool can be used in Breast MR to speed identification and investigation of enhancing regions.

The MultiKv tools provide support for Dual Energy and Spectral Imaging acquisitions from multiple vendors, providing standard image processing algorithms such as segmentation or contrast suppression, as well as generic toolkits for precise analysis and development of new techniques.

The embodiments described above can be applied to a variety of medical areas. For example, the techniques described above can be applied to vessel analysis (including Endovascular Aortic Repair (EVAR) and electrophysiology (EP) planning). Such vessel analysis is performed for interpretation of both coronary and general vessel analysis such as carotid and renal arteries, in addition to aortic endograft and electro-physiology planning. Tools provided as cloud services include auto-centerline extraction, straightened view, diameter and length measurements, Curved Planar Reformation (CPR) and axial renderings, as well as charting of the vessel diameter vs. distance and cross-sectional views. The vessel track tool provides a Maximum Intensity Projection (MIP) view in two orthogonal planes that travels along and rotates about the vessel centerline for ease of navigation and deep interrogation. Plaque analysis tools provide detailed delineation of non-luminal structure such as soft plaque, calcified plaque and intra-mural lesions.

In addition, the techniques described above can be utilized in the area of endovascular aortic repair. According to some embodiments, vascular analysis tools provided as cloud services support definition of report templates which captures measurements for endograft sizing. Multiple centerlines can be extracted to allow for planning of EVAR procedures with multiple access points. Diameters perpendicular to the vessel may be measured along with distances along the two aorto-iliac paths. Custom workflow templates may be used to enable the major aortic endograft manufactures' measurement specifications to be made as required for stent sizing. Sac segmentation and volume determination with a “clock-face” overlay to aid with documenting the orientation and location of branch vessels for fenestrated and branch device planning, may also be used. Reports containing required measurements and data may be generated.

The techniques described above can also be applied in the left atrium analysis mode, in which semi-automated left atrium segmentation of each pulmonary vein ostium is supported with a single-click distance pair tool, provided as cloud services, for assessment of the major and minor vein diameter. Measurements are automatically detected and captured into the integrated reporting system. These capabilities can be combined with other vessel analysis tools to provide a comprehensive and customized EP planning workflow for ablation and lead approach planning.

The techniques described above can also be utilized in calcium scoring. Semi-automated identification of coronary calcium is supported with Agatston, volume and mineral mass algorithms being totaled and reported on-screen. Results may be stored in an open-format database along with various other data relating to the patient and their cardiovascular history and risk factors. A customized report can be automatically generated, as part of cloud services, based upon these data. Also includes report generation as defined by the Society of Cardiovascular Computed Tomography (SCCT) guidelines.

The techniques described above can also be utilized in a time-volume analysis (TVA), which may include fully-automated calculation of left ventricular volume, ejection fraction, myocardial volume (mass) and wall thickening from multi-phasic data. A fast and efficient workflow provided as part of cloud services allows for easy verification or adjustment of levels and contours. The results are presented within the integrated reporting function.

The techniques described above can also be utilized in the area of segmentation analysis and tracking (SAT), which includes supports analysis and characterization of masses and structures in various scans, including pulmonary CT examinations. Features include single-click segmentation of masses, manual editing tools to resolve segmentation issues, automatic reporting of dimensions and volume, graphical 3D display of selected regions, integrated automated reporting tool, support for follow-up comparisons including percent volume change and doubling time, and support for review of sphericity filter results.

The techniques described above can also be utilized in the area of flythrough which may include features of automatic segmentation and centerline extraction of the colon, with editing tools available to redefine these centerlines if necessary. 2D review includes side-by-side synchronized supine and prone data sets in either axial, coronal or sagittal views with representative synchronized endoluminal views. 3D review includes axial, coronal and sagittal MPR or MIP image display with large endoluminal view and an unfolded view that displays the entire colon. Coverage tracking is supported to ensure 100% coverage with stepwise review of unviewed sections, one-click polyp identification, bookmark and merge findings, as well as a cube view for isolating a volume of interest and an integrated contextual reporting tool. Support is provided for use of sphericity filter results.

The techniques described above can also be utilized in the area of time-dependent analysis (TDA), which provides assessment tools for analyzing the time-dependent behavior of appropriate computerized tomographic angiography (CTA) and/or MRI examinations, such as within cerebral perfusion studies. Features include support for loading multiple time-dependent series at the same time, and a procedural workflow for selecting input and output function and regions of interest. An integrated reporting tool is provided as well as the ability to export the blood flow, blood volume and transit time maps to DICOM. The tools may also be used with time-dependent MR acquisitions to calculate various time-dependent parameters.

The techniques described above can also be utilized in the area of CTA-CT subtraction, which includes automatic registration of pre- and post-contrast images, followed by subtraction or dense-voxel masking technique which removes high-intensity structures (like bone and surgical clips) from the CTA scan without increasing noise, and leaving contrast-enhanced vascular structures intact.

The techniques described above can also be utilized in dental analysis, which provides a CPR tool which can be applied for review of dental CT scans, offering the ability to generate “panoramic” projections in various planes and of various thicknesses, and cross-sectional MPR views at set increments along the defined curve plane.

The techniques described above can also be utilized in the area of multi-phase MR (basic, e.g. breast, prostate MR). Certain MR examinations (for example, breast, prostate MR) involve a series of image acquisitions taken over a period of time, where certain structures become enhanced over time relative to other structures. This module features the ability to subtract a pre-enhancement image from all post-enhancement images to emphasize visualization of enhancing structures (for example, vascular structures and other enhancing tissue). Time-dependent region-of-interest tools are provided to plot time-intensity graphs of a given region.

The techniques described above can also be utilized in parametric mapping (e.g. for multi-phase Breast MR), in which the parametric mapping module pre-calculates overlay maps where each pixel in an image is color-coded depending on the time-dependent behavior of the pixel intensity. The techniques described above can also be utilized in the area of SphereFinder (e.g. sphericity filter for lung and colon). SphereFinder pre-processes datasets as soon as they are received and applies filters to detect sphere-like structures. This is often used with lung or colon CT scans to identify potential areas of interest. The techniques described can also be utilized in fusion for CT/MR/PET/SPECT. Any two CT, PET, MR or SPECT series, or any two-series combination can be overlaid with one assigned a semi-transparent color coding and the other shown in grayscale and volume rendering for anatomical reference. Automatic registration is provided and subtraction to a temporary series or to a saved, third series is possible.

The techniques described above can also be utilized in the area of Lobular Decomposition. Lobular Decomposition is an analysis and segmentation tool that is designed with anatomical structures in mind. For any structure or organ region which is intertwined with a tree-like structure (such as an arterial and/or venous tree), the Lobular Decomposition tool allows the user to select the volume of interest, as well as the trees related to it, and to partition the volume into lobes or territories which are most proximal to the tree or any specific sub-branch thereof. This generic and flexible tool has potential research applications in analysis of the liver, lung, heart and various other organs and pathological structures.

The techniques described above can also be utilized in the area of Volumetric Histogram. Volumetric Histogram supports analysis of a given volume of interest based on partition of the constituent voxels into populations of different intensity or density ranges. This can be used, for example, to support research into disease processes such as cancer (where it is desirable to analyze the composition of tumors, in an attempt to understand the balance between active tumor, necrotic tissue, and edema), or emphysema (where the population of low-attenuation voxels in a lung CT examination may be a meaningful indicator of early disease).

The techniques described above can also be utilized in the area of Motion Analytics. Motion Analytics provides a powerful 2D representation of a 4D process, for more effective communication of findings when interactive 3D or 4D display is not available. Any dynamic volume acquisition, such as a beating heart, can be subjected to the Motion Analysis, to generate a color-coded “trail” of outlines of key boundaries, throughout the dynamic sequence, allowing a single 2D frame to capture and illustrate the motion, in a manner that can be readily reported in literature. The uniformity of the color pattern, or lack thereof, reflects the extent to which motion is harmonic, providing immediate visual feedback from a single image.

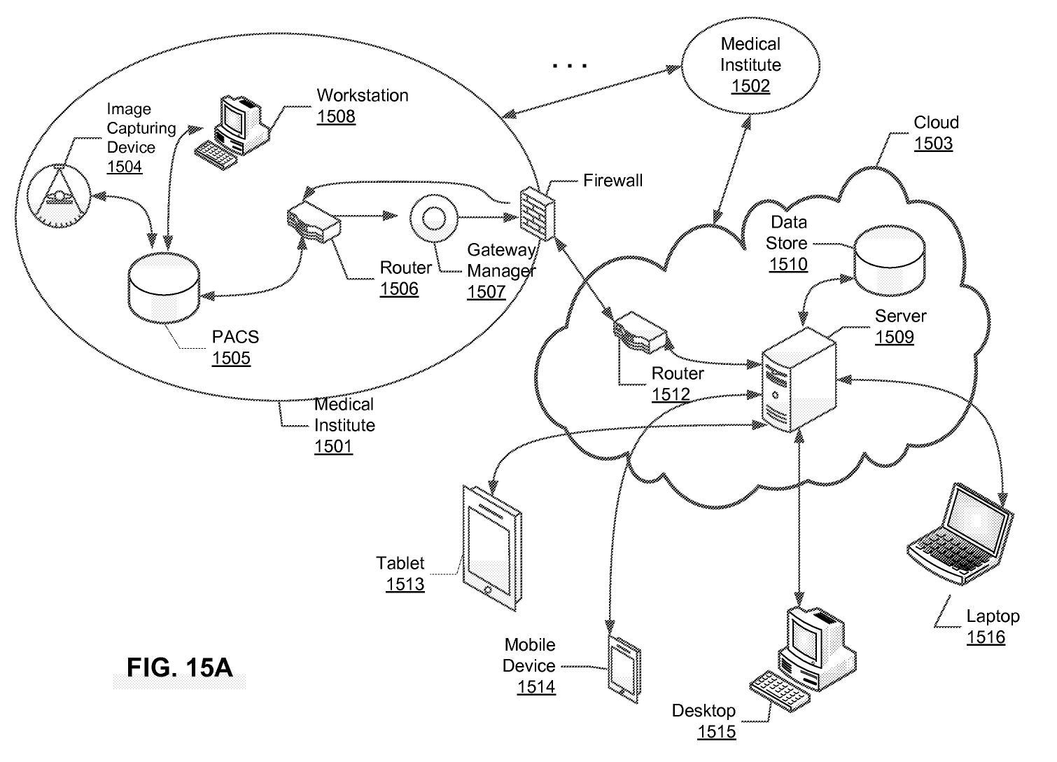

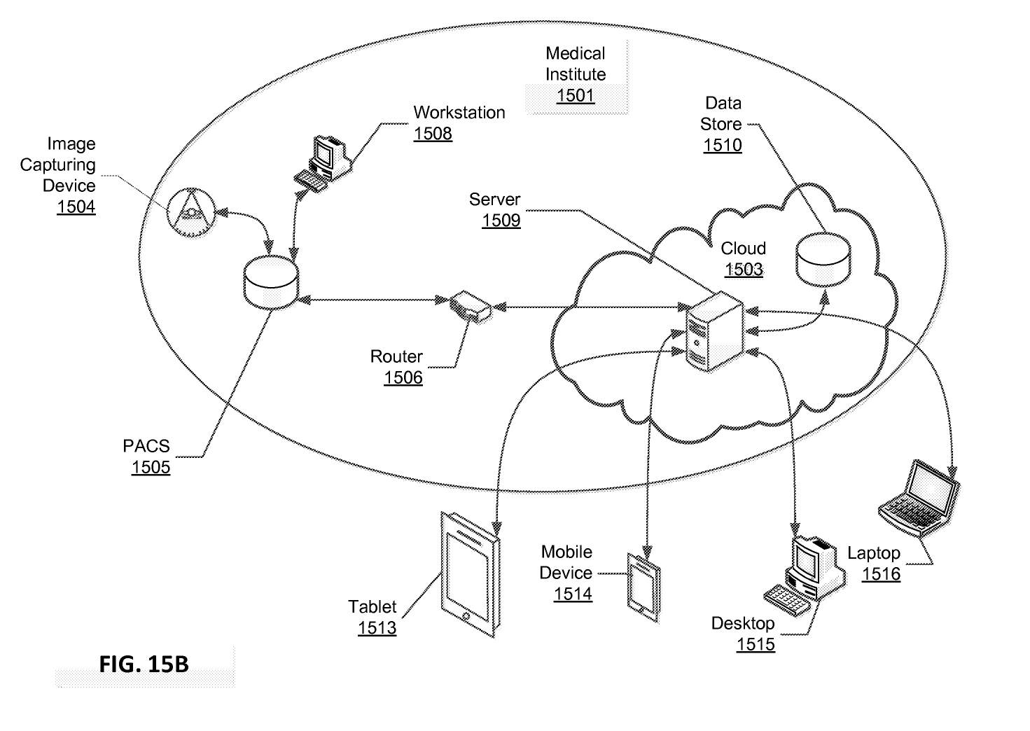

FIGS. 15A and 15B are block diagrams illustrating a cloud-based image processing system according to certain embodiments of the invention. For example, cloud server 1509 may represent medical information server 101 as described above. Referring to FIG. 15A, according to one embodiment, system 1500 includes one or more entities or institutes 1501-1502 communicatively coupled to cloud 1503 over a network. Entities 1501-1502 may represent a variety of organizations such as medical institutes having a variety of facilities residing all over the world. For example, entity 1501 may include or be associated with image capturing device or devices 1504, image storage system (e.g., PACS) 1505, router 1506, and/or data gateway manager 1507. Image storage system 1505 may be maintained by a third party entity that provides archiving services to entity 1501, which may be accessed by workstation 1508 such as an administrator or user associated with entity 1501. Note that throughout this application, a medical institute is utilized as an example of an organization entity. However, it is not so limited; other organizations or entities may also be applied.

In one embodiment, cloud 1503 may represent a set of servers or clusters of servers associated with a service provider and geographically distributed over a network. For example, cloud 1503 may be associated with a medical image processing service provider such as TeraRecon of Foster City, Calif. A network may be a local area network (LAN), a metropolitan area network (MAN), a wide area network (WAN) such as the Internet or an intranet, or a combination thereof. Cloud 1503 can be made of a variety of servers and devices capable of providing application services to a variety of clients such as clients 1513-1516 over a network. In one embodiment, cloud 1503 includes one or more cloud servers 1509 to provide image processing services, one or more databases 1510 to store images and other medical data, and one or more routers 1512 to transfer data to/from other entities such as entities 1501-1502. If the cloud server consists of a server cluster, or more than one server, rules may exist which control the transfer of data between the servers in the cluster. For example, there may be reasons why data on a server in one country should not be placed on a server in another country.

Server 1509 may be an image processing server to provide medical image processing services to clients 1513-1516 over a network. For example, server 1509 may be implemented as part of a TeraRecon AquariusNET™ server and/or a TeraRecon AquariusAPS server. Data gateway manager 1507 and/or router 1506 may be implemented as part of a TeraRecon AquariusGATE device. Medical imaging device 1504 may be an image diagnosis device, such as X-ray CT device, MRI scanning device, nuclear medicine device, ultrasound device, or any other medical imaging device. Medical imaging device 1504 collects information from multiple cross-section views of a specimen, reconstructs them, and produces medical image data for the multiple cross-section views. Medical imaging device 1504 is also referred to as a modality.

Database 1510 may be a data store to store medical data such as digital imaging and communications in medicine (DICOM) compatible data or other image data. Database 1510 may also incorporate encryption capabilities. Database 1510 may include multiple databases and/or may be maintained by a third party vendor such as storage providers. Data store 1510 may be implemented with relational database management systems (RDBMS), e.g., Oracle™ database or Microsoft® SQL Server, etc. Clients 1513-1516 may represent a variety of client devices such as a desktop, laptop, tablet, mobile phone, personal digital assistant (PDA), etc. Some of clients 1513-1516 may include a client application (e.g., thin client application) to access resources such as medical image processing tools or applications hosted by server 1509 over a network. Examples of thin clients include a web browser, a phone application and others.

According to one embodiment, server 1509 is configured to provide advanced image processing services to clients 1513-1516, which may represent physicians from medical institutes, instructors, students, agents from insurance companies, patients, medical researchers, etc. Cloud server 1509, also referred to as an image processing server, has the capability of hosting one or more medical images and data associated with the medical images to allow multiple participants such as clients 1513-1516, to participate in a discussion/processing forum of the images in a collaborated manner or conferencing environment. Different participants may participate in different stages and/or levels of a discussion session or a workflow process of the images.

According to some embodiments, data gateway manager 1507 is configured to automatically or manually transfer medical data to/from data providers (e.g., PACS systems) such as medical institutes. Such data gateway management may be performed based on a set of rules or policies, which may be configured by an administrator or authorized personnel. In one embodiment, in response to updates of medical images data during an image discussion session or image processing operations performed in the cloud, the data gateway manager is configured to transmit over a network (e.g., Internet) the updated image data or the difference between the updated image data and the original image data to a data provider such as PACS 1505 that provided the original medical image data. Similarly, data gateway manager 1507 can be configured to transmit any new images and/or image data from the data provider, where the new images may have been captured by an image capturing device such as image capturing device 1504 associated with entity 1501. In addition, data gateway manager 1507 may further transfer data amongst multiple data providers that is associated with the same entity (e.g., multiple facilities of a medical institute). Furthermore, cloud 1503 may include an advanced preprocessing system (not shown) to automatically perform certain pre-processing operations of the received images using certain advanced image processing resources provided by the cloud systems. In one embodiment, gateway manager 1507 is configured to communicate with cloud 1503 via certain Internet ports such as port 80 or 443, etc. The data being transferred may be encrypted and/or compressed using a variety of encryption and compression methods. The term “Internet port” in this context could also be an intranet port, or a private port such as port 80 or 443 etc. on an intranet.

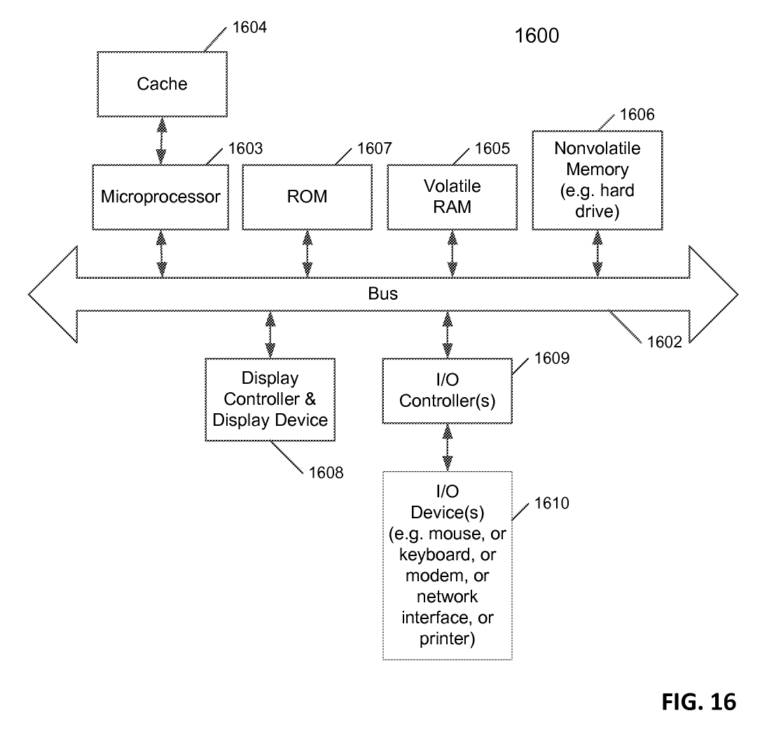

FIG. 16 is a block diagram of a data processing system, which may be used with one embodiment of the invention. For example, the system 1600 may be used as part of a server or a client as described above. For example, system 1600 may represent medical information server 101 described above, which is communicatively coupled to a remote client device or another server via network interface 1610. At least ECCD engine 103, medical image processing system 210, and medical data integrator 205, as described above, may be hosted by system 1600.

Note that while FIG. 16 illustrates various components of a computer system, it is not intended to represent any particular architecture or manner of interconnecting the components; as such details are not germane to the present invention. It will also be appreciated that network computers, handheld computers, cell phones and other data processing systems which have fewer components or perhaps more components may also be used with the present invention.

As shown in FIG. 16, the computer system 1600, which is a form of a data processing system, includes a bus or interconnect 1602 which is coupled to one or more microprocessors 1603 and a ROM 1607, a volatile RAM 1605, and a non-volatile memory 1606. The microprocessor 1603 is coupled to cache memory 1604. The bus 1602 interconnects these various components together and also interconnects these components 1603, 1607, 1605, and 1606 to a display controller and display device 1608, as well as to input/output (I/O) devices 1610, which may be mice, keyboards, modems, network interfaces, printers, and other devices which are well-known in the art.

Typically, the input/output devices 1610 are coupled to the system through input/output controllers 1609. The volatile RAM 1605 is typically implemented as dynamic RAM (DRAM) which requires power continuously in order to refresh or maintain the data in the memory. The non-volatile memory 1606 is typically a magnetic hard drive, a magnetic optical drive, an optical drive, or a DVD RAM or other type of memory system which maintains data even after power is removed from the system. Typically, the non-volatile memory will also be a random access memory, although this is not required.

While FIG. 16 shows that the non-volatile memory is a local device coupled directly to the rest of the components in the data processing system, the present invention may utilize a non-volatile memory which is remote from the system; such as, a network storage device which is coupled to the data processing system through a network interface such as a modem or Ethernet interface. The bus 1602 may include one or more buses connected to each other through various bridges, controllers, and/or adapters, as is well-known in the art. In one embodiment, the I/O controller 1609 includes a USB (Universal Serial Bus) adapter for controlling USB peripherals. Alternatively, I/O controller 1609 may include an IEEE-1394 adapter, also known as FireWire adapter, for controlling FireWire devices.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The techniques shown in the figures can be implemented using code and data stored and executed on one or more electronic devices. Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals—such as carrier waves, infrared signals, digital signals).

The processes or methods depicted in the preceding figures may be performed by processing logic that comprises hardware (e.g. circuitry, dedicated logic, etc.), firmware, software (e.g., embodied on a non-transitory computer readable medium), or a combination of both. Although the processes or methods are described above in terms of some sequential operations, it should be appreciated that some of the operations described may be performed in a different order. Moreover, some operations may be performed in parallel rather than sequentially.

In the foregoing specification, embodiments of the invention have been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

1-20. (canceled)

21. A medical information system, comprising:
a plurality of data sources, each data source storing medical data;
a client device; and,
a medical information server, comprising:
data source interfaces to each of the plurality of data sources, and
an evolving contextual clinical data engine, the evolving contextual clinical data engine performing functions in response to user input from a user on the client device, the functions including:
determining what types of medical data the user is likely interested in based on the user input and additionally based on evolving contextual clinical data rules or modules that are modeled based on prior user interactions with the user or other users, and
mining data objects from more than one of the plurality of data sources and integrating the mined data objects based on common metadata of the data objects;
wherein the medical information server provides to the user, medical data results based on the mined and integrated data objects.

22. A medical information system as in claim 21, wherein plurality of data sources include more than one of the following:
Laboratory Information System (LIS);
Radiology Information System (RIS);
Enterprise Content Management Systems (ECM);
Electronic Medical Record (EMR);
Hospital Information System (HIS);
Picture Archiving and Communication System (PACS);
Vendor Neutral Archive (VNA);
EMR data;
Health Information Exchange (HIE) servers.

23. A medical information system as in claim 21, wherein the common metadata is at least one of the following:
patient identification;
accession number;
data;
time frame;
body part;
body area;
medical condition;
procedure;
symptom.

24. A medical information system as in claim 21, wherein the medical information server provides the medical data results to the user by forwarding to the client device the medical data results for displaying on a display within the client device.

25. A medical information system as in claim 21, wherein the medical information server provides the medical data results to the user by storing the medical data results for later access by the user.

26. A medical information system as in claim 21, wherein the medical information server provides the medical data results to the user by printing the medical data results.

27. A medical information system as in claim 21, wherein the functions performed by the evolving contextual clinical data engine additionally include:
analyzing the medical data results to generate recommendations for further action by the user; and,
communicating the recommendations to the user.

28. A medical information system as in claim 27, wherein analyzing the medical data results to generate recommendations for further action by the user, the analysis includes analyzing information from a plurality of patients.

29. A medical information system as in claim 21, wherein the medical information server additionally comprises a data integrator that integrates medical data retrieved from the plurality of data sources into one or more views of medical information.

30. A medical information system as in claim 21, wherein the medical information server additionally comprises a medical image processing system that performs one or more image processing operations on at least one medical image included in the data objects mined from the more than one of the plurality of data sources.

31. A medical information system as in claim 21, wherein the evolving contextual clinical data engine includes:
a user behavioral analyzer that accesses a user database to analyze user interaction history; and,
a rule engine that compiles and generates the contextual clinical data rules based at least partially on the user interaction history analyzed by the user behavior analyzer.

32. A medical information system as in claim 21, wherein the functions performed by the evolving contextual clinical data engine additionally include:
analyzing tracked click through rates and view times as monitored by a user data collector.

33. A medical information system as in claim 21, wherein the functions performed by the evolving contextual clinical data engine additionally include:
analyzing total interpretation time for at least one of the following:
time that the user views multiple related images or information objects;
time that the user performs advanced image processing steps;
total time that the user reviews information relation got a single patient.

34. A medical information system as in claim 21, wherein the medical information server additionally comprises a data integrator that integrates medical data retrieved from the plurality of data sources into one or more views of medical information, the data integrator including:
a data retrieval module;
a view generator;
a user action analysis module; and,
medical data interface modules.

35. A medical information system, comprising:
a plurality of data sources, each data source storing medical data; and,
a medical information server, comprising:
an evolving contextual clinical data engine, the evolving contextual clinical data engine determining what types of medical data the user is likely interested in based on the user input and additionally based on evolving contextual clinical data rules or modules that are modeled based on prior user interactions with the user or other users, and
a data integrator, the data integrator including:
a user action analysis module the analyzes user actions performed by a user of a client device, the user actions including user requests,
a data retrieval module that in response to a request from a user determines one or more of the data sources that provide responsive medical data that is responsive to the request, the responsive medical data including information directly requested by the request, and the responsive medical data including data that the evolving contextual clinical data engine determines are likely of interest to the user,
medical data interface modules that interface with the plurality of data sources, and
a view generator that generates one or more views of the responsive medical data.

36. A medical information system as in claim 35, wherein plurality of data sources include more than one of the following:
Laboratory Information System (LIS);
Radiology Information System (RIS);
Enterprise Content Management Systems (ECM);
Electronic Medical Record (EMR);
Hospital Information System (HIS);
Picture Archiving and Communication System (PACS);
Vendor Neutral Archive (VNA);
EMR data;
Health Information Exchange (HIE) servers.

37. A medical information system as in claim 35, wherein the data integrator additionally includes:
an analysis result/recommendation block that receives from the evolving contextual clinical data engine recommendations for further action by the user.

38. A medical information system as in claim 35, wherein the functions performed by the evolving contextual clinical data engine additionally analyzes the medical data results to generate recommendations for further action by the user.

39. A medical information system as in claim 38, wherein analyzing the medical data results to generate recommendations for further action by the user, the analysis includes analyzing information from a plurality of patients.

40. A medical information system as in claim 35, wherein the medical information server additionally comprises a medical image processing system that performs one or more image processing operations on at least one medical image included in the data objects mined from the more than one of the plurality of data sources.
